(19) United States
(12) Patent Application Publication
(10) Pub. No.: US 2014/0088575 A1
(43) Pub. Date: Mar. 27, 2014

(54) DEVICES FOR EFFECTIVE AND UNIFORM DENERVATION OF NERVES AND UNIQUE METHODS OF USE THEREOF

(71) Applicant: Trimedyne, Inc., Irvine, CA (US)

(72) Inventor: Marvin P. Loeb, Laguna Woods, CA (US)

(21) Appl. No.: 14/039,800

(22) Filed: Sep. 27, 2013

Related U.S. Application Data
(60) Provisional application No. 61/706,531, filed on Sep. 27, 2012.

(51) Int. Cl. A61B 18/24; A61B 18/22

(52) U.S. Cl. CPC A61B 18/245; A61B 18/22
USPC 606/7; 606/16

(57) ABSTRACT

Apparatus for delivering laser energy suitable for denervation, such as renal denervation and the like, comprises an optical fiber inside a cannula and defining a channel therebetween for delivery of a liquid to cool and clean the tip of the optical fiber and to cool tissue subjected to laser irradiation, while the apparatus is Stationed, Moved, Rotated and or Swept during the emission of laser energy.

### CROSS-REFERENCE TO RELATED APPLICATION

This application claims the benefit of U.S. Provisional Application No. 61/706,531, filed on Sep. 27, 2012, which is incorporated herein by reference in its entirety.

### FIELD OF THE INVENTION

This invention relates to devices and treatment of medical conditions, such as hypertension, asthma, Type II diabetes, obesity, cardiac arrhythmia, and a host of other conditions, which are not of bacterial or viral origin and have not been conclusively identified as genetic, hereditary, diet or environmentally related. In one aspect this invention relates to denervation of malfunctioning sympathetic, parasympathetic and sensory nerves.

### BACKGROUND OF THE INVENTION

Many people suffer from medical conditions which do not arise from a bacterial or viral infection and have not been conclusively linked to a genetic, hereditary, environmental, dietary or other cause. The treatment of such conditions, which affect many millions of people in the United States and hundreds of millions in other countries, are costly in lives and a significant cost to the healthcare system. Some of these conditions, such as hypertension, asthma and Type II diabetes, are treated with medications, but the condition cannot be completely relieved in many patients. Other conditions, such as arrhythmia and obesity, are treated with surgery or a medical device. For example, obesity is being treated by removal of part of the stomach or the application of a lap band, to cause food intake to be reduced and safety or the feeling of fullness to be sensed, and cardiac arrhythmia is treated with a pacemaker or the interior surface of the atrium or ventricle can be cut or coagulated in a “maze” procedure to interrupt nerves that cause arrythmia.

Surgical procedures entail a high cost with an associated recuperation period and mortality rate, and pharmaceuticals can have interactions with other drugs, entail a variety of adverse effects and are expensive.

The body's immune system sometimes cannot cope with a bacterial or viral infection or a colony of malignant cells whose growth cannot be stopped, the digestive system allows too much cholesterol and low density lipoproteins to enter the circulatory system, the kidneys fail to regulate blood pressure properly, resulting in hypertension, the bronchi of the lung sometimes constrict unnecessarily, causing an asthma attack, even if no smoke or toxic irritants are in the air, and the nervous system has a multitude of failures that result in overeating and obesity, epilepsy, pychosis, anxiety, depression, schizophrenia, seizures, Parkinson's disease, senile dementia and many others.

Some of these conditions are accepted as inevitable consequences, and patients rely upon the therapies and drugs that are available, despite their cost, adverse effects and risk of death. It is an object of the present invention to safely and effectively treat these medical conditions which, as mentioned above, arise from as yet unidentified causes, with minimally invasive, thermal energy devices and procedures.

Even if nerves function properly, if the underlying cause, for example, physical damage or arthritis, cannot be easily treated or is untreatable, interrupting the pain signals can bring relief to the patient. Elimination of the pain signals may also help relieve the condition by reducing inflammation at the site of the pain. If nerves malfunction and create the sensation of pain, when there is no cause for the pain, interrupting the pain signals can eliminate the patient's pain.

The function of nerves can be determined by a process called Evoked Potential. For example, electrically stimulating nerves with an electromyograph can be detected by their effect on muscle cells, glands and other tissues.

Malfunctioning nerves can be coagulated or vaporized.

CTH:YAG or “Holmium” lasers and fiber-optic laser energy delivery devices are commercially available from the owner of this application, Trimedyne, Inc. (“Trimedyne”), Lake Forest, Calif. 92630, for treating a variety of medical conditions. One of these conditions is the treatment of herniated spinal discs in minimally invasive, outpatient procedures, by applying Holmium laser energy to vaporize a portion of the excess, benign growth of the nucleus pulposa tissue of a spinal disc. This excess growth of tissue causes the disc to bulge and press upon nerves in the spinal column, causing unrelenting pain. These procedures have been shown in a number of published papers to produce success rates, based upon recognized pain score criteria, of 84% to 95% in hundreds of patients for up to two years.

If the above herniated disc treatment fails to relieve the back, leg or nerve pain, the pain may arise, for example, in the zypgsophyseal or “facet” joints of the vertebra or the partially fused joint of the sacrum and the ilium of the hip, called the “sacroiliac” joint.

Trimedyne's Holmium lasers and fiber-optic devices have been used in minimally invasive, outpatient procedures, to denervate nerve endings in the capsule of the facet joints of the vertebra to end the transmission of pain signals to the brain, with an average of 71% of the patients having at least a 50% reduction in back pain at 3 to 6 years.

We recently learned that a diagnosis of arthritis of the facet joints by an imaging study does not often correlate with back pain. Some patients with extensive arthritis by MRI, CT scan or x-ray imaging may not suffer significant pain, and some patients with minimal or no arthritis by MRI, CT or x-ray imaging may suffer severe pain.

Based on this information, we deduced that the cause of these anomalies is the malfunctioning of sympathetic (“S”) nerves and/or parasympathetic (“PS”) nerves of the facet joints, which either failed to send pain signals to the brain when severe arthritis was present or sent false pain signals to the brain when arthritis was minimal. S nerves and PS nerves are individually or collectively defined in this Specification and the Claims as “S/PS” nerves. S/PS nerves are a part of the autonomic nervous system.

We then began to identify S/PS nerves whose malfunction could cause medical conditions which did not arise from a bacterial or viral infection, or are not conclusively linked to a genetic, hereditary, dietary, environmental or other cause, which conditions might be treated, reversed, reduced or prevented by vaporizing or coagulating a sufficient length of these nerves to significantly interrupt messages to or from the brain from malfunctioning S/PS nerves.

We also considered S/PS nerves whose malfunction could result in the constriction or relaxation of smooth, circular, striated or other muscle cells.

S/PS nerves usually run longitudinally in parallel through the outer layer, on the exterior of or alongside the walls of blood vessels. S/PS nerves also run longitudinally through layers of muscle cells, solid organs, hollow organs, glands or ducts and through or alongside bones and other tissues. S/PS nerves are indistinguishable from each other visually. For example, S/PS nerves also run longitudinally through the second layer and, to a lesser extent, through the fourth layer of the bronchi of the lungs, and S/PS nerves run through the outer layer of, on the exterior of or alongside arteries and veins, such as the renal arteries and vagus nerve, the stomach wall, duodenum, colon, liver and pancreas of the digestive system.

S/PS nerves are further divided into efferent S/PS nerves, which carry signals to the brain, and afferent S/PS nerves that carry signals from the brain. S/PS nerves regenerate or grow back together if damaged or cut. As a result, a significant area or volume of malfunctioning S/PS nerves in, on the exterior of or alongside an artery, vein, organ, gland, layer of muscle cells, elastic fiber layers, connective tissue layers or other tissues must be vaporized or coagulated to interrupt their transmission of signals to or from the brain.

While nerves have been killed or temporarily deactivated for many years with injections of a variety of agents, as known in the art, to stop the patient from suffering pain, so far as we know, nobody has deduced what we have discovered: that efferent and afferent S/PS nerves can malfunction and either fail to send signals to the brain or send false signals to the brain, blood vessels, bronchi, ducts, glands, organs, spinal discs, vertebra, joints, connective tissue, a layer of muscle cells, an elastic fiber layer or other tissues.

Some sensory nerves (“SN” nerves) of the somatic nervous system can also malfunction in the same manner as S/PS nerves. Even if SN nerves are functioning normally, if the cause of the pain is difficult to treat or is untreatable, interrupting pain signals from SN nerves may give the patient relief from the pain. Doing so may also help relieve the cause of the pain, by reducing inflammation resulting from nerve excitation. S, PS and SN nerves are individually or collectively defined in this Specification and the Claims as “S/PS/SN” nerves.

S/PS/SN nerves and tissues containing S/PS/SN nerves, layers of elastic fibers, and their membranes, blood vessels, ducts, organs, glands, bones, joints, tendons, valves, sphincters, collagen bearing tissues and other tissues are individually or collectively defined in this Specification and referred to in the Claims as a “Target NerveTissue” or “Target Nerve Tissues”.

It may not be necessary or desirable to denervate all of the S/PS/SN nerves of a Target Nerve Tissue. It may only be necessary to reduce the volume of S/PS/SN nerve signal traffic to the brain to a level at which the brain does not react to the pain signals, false pain signals or the absence of pain signals. It follows that denervating ganglia or bundles of S/PS/SN nerves, even small bundles, can significantly reduce the volume of S/PS/SN nerve signal traffic.

For example, if the condition perceived by the brain requires the blood vessels of the kidney or the bronchi of the lungs to be constricted, the rate of compression of the atria of the heart to be increased or decreased, or the output of a specific hormone, enzyme or other substance produced by glands or one or more of the digestive organs to be increased or decreased, even if these conditions are actually normal, the brain acts to correct the perceived abnormal condition, which may result, for example, in high blood pressure or hypertension, asthma, an arrhythmia, Type II diabetes, obesity and a host of other medical conditions.

The status or condition of Target Nerve Tissues can be altered by thermal energy. Target Nerve Tissues can be frozen by subjecting them to a cold gas at a temperature of 0° C. or lower, preferably by a cryogenically cooled gas at temperatures substantially lower than 0° C.

Target Nerve Tissues can be denatured by subjecting them to temperatures of about 55° C. to 60° C., at which temperatures certain proteins and the DNA of the cells of the Target Nerve Tissue are damaged, inhibiting or prohibiting their replication or re-growth. Target Nerve Tissues can be coagulated by subjecting them to temperatures of about 62° C. to 99° C., which breaks-down cell walls and the cells' contents, as well as coagulating blood in blood vessels, depriving the cells of oxygen and nutrients and causing them to die.

Target Nerve Tissues can also be ablated or vaporized by subjecting them to temperatures of 100° C. or more, at which temperatures water in the cells is turned to steam, the cells' membranes and their contents are turned to gasses and the vaporized tissue visually disappears. However, vaporizing Target Nerve Tissues at temperatures at or above 100° C. can damage normal, adjacent tissues, so vaporization of Target Nerve Tissues must be carefully controlled.

The use of thermal energy to alter Target Nerve Tissues by freezing, denaturing, coagulating or vaporizing them are individually or collectively defined in this Specification and referred to in the Claims as to “Alter” or “Altering” a Target Nerve Tissue or Target Nerve Tissues.

Altering Target Nerve Tissues can be achieved by delivery of various forms of thermal energy, including pulsed laser energy, continuous wave laser energy, pulsed intense incoherent light, continuous wave intense incoherent light, electrically generated thermal energy (such as from an electric arc, electrical impedance or resistance, piezo electric, electro-shock wave or ESW, radiofrequency (“RF”), microwave (“MW”) or ultrasound (US) energy), the insertion of one or more needles, each containing an optical fiber for delivery of laser energy to a desired depth within a Target Nerve Tissue, focusing multiple beams of laser, x-ray, photons, RF, microwave or ultrasound energy to intersect at a desired point in a Target Nerve Tissue (with minimal adverse effect from each individual beam of energy on intervening tissues), a sterile, biocompatible heated liquid (such as water or saline), a cold or cryogenically cooled sterile, biocompatible gas, (such as CO2 or nitrogen gas), and other types of thermal energy, are individually or collectively referred to in this Specification and the Claims as “Thermal Energy” or “Thermal Energies.”

Since S/PS/SN nerves may regenerate or re-grow if denatured, coagulated or vaporized, a means to uniformly and completely interrupt their signals is preferred. An electrically or x-ray based Source of Thermal Energy, such as those described above, emits Thermal Energy continuously, not allowing time for the tissue to cool. Also, electrically based Thermal Energy does not produce uniform or complete interruption of S/PS/SN nerves, as many electrically based Sources of Thermal Energy tend to follow and dissipate within pathways through tissue with greater salinity (conductivity), such as blood in blood vessels.

Hot gasses or liquids, continuous wave intense light and continuous wave laser energy do not allow time for a Target Nerve Tissue to cool and cause thermal damage by heat conduction or diffusion to adjacent tissues. US and MW energy is also usually continuous wave and passes through a Target Nerve Tissue to a different extent, based on the density of the tissue, resulting in an erratic effect. Cooled or cryogenically cooled sterile, biocompatible gasses cannot often be precisely delivered and maintained in place for a sufficient period of time to effectively alter many Target Nerve Tissues.

While some continuous wave thermal energy can be gated or pulsed by turning the Source of Thermal Energy “on” and “off” or periodically interrupting it with an impenetrable barrier, the amount of Thermal Energy delivered is reduced. For example, if the Source of Thermal Energy is “on” for one second and “off” for one second, to allow time for the tissue to cool, the amount of Thermal Energy delivered to a Target Nerve Tissue is reduced by 50%.

If the Thermal Energy is “on” for one second and “off” for nineteen seconds, the amount of Thermal Energy delivered to a Target Nerve Tissue is reduced by 95%. Thus, to produce 20 watts of energy for one second, with 19 seconds for the tissue to cool, would require a 400 watt laser, which could be costly. Rapidly pulsed RF energy usually raises the temperature of tissue to only about 47° C., rendering it incapable of effectively altering a Target Nerve Tissue, unless very high power RF generators are used, which could be unsafe.

This is not true in the case of certain pulsed lasers, such as Excimer lasers, Chromium, Thulium, Holmium:YAG lasers (often referred to as “CTH:YAG” lasers or simply as “Holmium” lasers), Erbium:YAG lasers, CO₂ lasers and other pulsed lasers, all of which deliver very short, very high peak power pulses of laser energy. Of these Sources of laser energy, Excimer, Erbium and CO₂ lasers require high hydroxyl ion content optical fibers, ultra-low hydroxyl ion content optical fibers or hollow, silver internally coated optical fibers, respectively, which are expensive, and the ability of Excimer, Erbium and CO2 lasers to deliver laser energy through such optical fibers is usually limited to about 10 watts.

Also, the light extinction depth of Excimer, Erbium:YAG and CO2 lasers is very short (only 5 to 50 microns) and may not reach sufficiently far into a Target Nerve Tissue to alter S/PS/SN nerves, layers of muscle cells or other Target Nerve Tissues. CTH:YAG or “Holmium” laser energy penetrates tissue to a depth of 0.4 millimeters or 400 microns, making Holmium lasers ideal for treating many Target Nerve Tissues of various Medical Conditions of Patients.

For example, a Holmium laser, producing light energy at a wavelength of 2100 nm, a wavelength of light which is highly absorbed by water, a constituent of all cells, can generate an average of power of up to 100 watts or more of energy in pulses of 350 microseconds in duration. At a pulse repetition rate of 10 pulses per second (“Hertz”), a second consists of ten segments of 100,000 microseconds. After each 350 microsecond pulse of Holmium laser energy, there are 99,650 microseconds for the tissue to cool, until the next laser energy pulse occurs. As a result, coagulation and charring of adjoining tissues is largely avoided, reducing edema and often hastening healing.

While a Holmium laser may be rated at 100 watts of average power, each pulse at 100 watts of power, at a pulse repetition rate of ten pulses per second, can reach a peak power of about 9,000 watts, effectively altering by almost instant vaporization any tissue in its path, up to the 0.4 mm light extinction depth in tissue of the Holmium laser's 2100 nm wavelength of light.

Even with thermal diffusion, in a fluid field, consisting of sterile water or saline, at an energy level of about 20 watts over a laser energy emission period of about 15 seconds, Holmium laser energy's aggregate thermal effect on a Target Nerve Tissue is only about 1 mm in depth.

The short, 0.4 mm tissue penetration depth and short, 350 microsecond, very high peak power pulses of Holmium lasers provide the ability to precisely and effectively Alter most Target Nerve Tissues, with time between pulses for the tissue to cool, without damage to adjacent tissues, including blood vessels, ducts and other nerves.

Diode, KTP and Nd:YAG lasers, for example, which produce continuous or near-continuous wave energy, penetrate tissue to their light extension depth of about 2 to 4 mm. With thermal diffusion, about 20 watts of laser energy of these lasers emitted during 15 seconds of emission, generally penetrate tissue to an aggregate depth of about 5 to 8 mm, several times deeper than Holmium laser energy, and do not allow time for the tissue to cool.

However, if the Target Nerve Tissue is deeper than about 1-2 mm, to avoid thermal damage to intervening tissues, (a) the Source of Thermal Energy can be selected based on its light or thermal extinction depth in a particular type, color and density of a Target Nerve Tissue, (b) multiple beams of Thermal Energy, converging at a depth in tissue at which an aggregation of S/PS/SN nerves of a Target Nerve Tissue is present, or (c) one or more needles, each containing an optical fiber to transmit laser energy, may be inserted into tissue to a depth at which an aggregation of S/PS/SN nerves of a Target Nerve Tissue occurs.

Our deduction that malfunctioning S/PS/SN nerves of the renal arteries can create hypertension was recently confirmed in a randomized, controlled, 106 patient, multicenter clinical trial of a radiofrequency (RF) catheter made by Ardian, Inc. of Palo Alto, Calif. (“Ardian”). This clinical trial was conducted outside the United States.

Ardian's RF catheter was inserted into the main renal arteries, before their branching into smaller renal arteries, in a percutaneous intra-luminal procedure, like balloon angioplasty, to denervate S/PS/SN nerves in the adventitia or outermost layer and on the exterior of the renal arteries to treat hypertension (systolic blood pressure of 40 mm of mercury) resistant to drug therapy. An aggregate of about 5,760 joules of RF energy was emitted at about six spots (about 960 joules at each spot) within each of the main renal arteries. This clinical trial compared a group of 52 RF treated patients, who also received optimal drug therapy, to a group of 54 control patients, who received only optimal drug therapy, over a six month period.

There was no significant change in systolic blood pressure in the 54 control patients, but in the 52 RF treated patients, after application of RF energy at a series of points to the walls of the main renal arteries, the levels of rennin and Angiotensin II (vaso-constrictors) in the bloodstream fell by 40% to 50% and, at six months, the systolic blood pressure of the RF treated group was reduced by an average of 33 mm of mercury (Hg), compared to that of the control group (Eisler, M. et al., Renal sympathetic denervation in patients with treatment-resistant hypertension (The Symplicity HTN-2 Trial): a randomized controlled trial. Lancet. 2010, Dec. 4, 376(9756):1903-09).

While an admirable benefit, this reduction in systolic blood pressure is not sufficient to normalize a drug resistant hypertensive patient with a systolic blood pressure of 175 to 300 mm Hg.

Also, our deduction that malfunctioning S/PS/SN nerves of the bronchi of the lungs can create asthma was recently confirmed in a 288 adult patient, randomized, controlled clinical trial of an RF catheter by Asthmatx, Inc. of Sunnyvale, Calif. Asthmatx's RF catheter was used through a bronchoscope to denature (preventing the replication of muscle cells in the third or medial (middle) layer of the bronchi) and thin the muscle cell layer by coagulation of the muscle cells.

A high level or RF energy was emitted at a series of about 70 points in the bronchi in three separate procedures, about three weeks apart. Separating the therapy into three procedures was necessary because pain during and after the procedure was significant, due to thermal damage to the sensitive, inner, endothelial cell layer of the bronchi. After allowing for drop-outs, this clinical trial compared 173 RF treated to 95 sham treated, control patients over a one year period. Both groups received optimal medical (drug) therapy throughout the trial.

The RF treated group had an average of 32 fewer severe asthma episodes, but a slightly higher number of adverse events and hospitalizations than the sham control group, over a period of one year (Castro, M. et al., Effectiveness and Safety of Bronchial Thermoplasty in the Treatment of Severe Asthma. Am. J. Respir. Crit. Care Med., Vol 181; 116-121,2010).

While the authors of the above paper attributed the pain to the bronchoscope, and said the reduction in asthma episodes was due to coagulation or “thinning” of muscle cells in the third or middle layer of the bronchi, we believe the reduction in asthma episodes was mainly or at least partially due to coagulation of S/PS/SN nerves in the second layer and, to a lesser extent, the fourth layer of the bronchi.

We believe the amount of RF energy delivered to coagulate muscle cells in the middle layer of the bronchi may have been in excess of the amount needed to produce about the same reduction of asthma episodes that could occur from simply denervating and interrupting a sufficient area or volume of S/PS/SN nerves in the second layer and some in the fourth layer of the bronchi at up to three or more spots in each of the main, left and right branches of the bronchi. Such excess RF energy may have contributed to the pain that required the therapy to be delivered in three separate procedures.

In a recent clinical study of the Holmium laser and optical fiber devices made by the owner of this application in the denervation of S/PS/SN nerves in the facet joints of the vertebra, 194 patients were treated with a burr to debride or grind-off the outer layer of the capsules of the facet joints of the vertebra, and laser energy was used to coagulate and denervate the exposed nerve endings of the facet joints. At their last visit at three or six years after the therapy, an average of 71% of the patients had at least a 50% reduction of back pain (Haufe S M W and Mork A R, Endoscopic Facet Debridement for the treatment of facet arthritic pain—a novel new technique. Int. J. Med. Sci. 2010, 7:120-123).

By comparison, in an abstract of a paper on a clinical study of 93 patients in which RF energy was used to denervate the S/PS/SN nerve endings in the facet joints of the vertebra, only 50% of the patients had significant pain relief immediately after the RF therapy, only 38% had significant pain relief at 3 months and only 25% of the patients had significant pain reduction at 73 months after the RF therapy (Jerosch J et al. Long-Term results following percutaneous facet coagulation (Abstract: Z Orthop Ihre Grenzgeb, May-June 1993, (3):24′-7). While the Abstract was published in English, this paper was published in German.

As shown by the aforementioned papers on facet joint nerve denervation and for the reasons described above, we believe Holmium laser energy will more uniformly and completely Alter and more effectively interrupt nerve signals to and from malfunctioning S/PS/SN nerves, with a longer lasting effect than RF energy.

The process of using thermal energy to Alter S/PS/SN nerves to prevent their malfunctioning or functioning is defined in this Specification and the Claims as “Denervating” or to “Denervate” S/PS/SN nerves. Since S/PS/SN nerves may regenerate or grow back together, a sufficient volume or area of the S/PS/SN nerves or tissue containing S/PS/SN nerves must be Denervated to create a gap sufficiently large to prevent or significantly delay the S/PS/SN nerves from growing back together, interrupting their transmissions of pain signals to the brain, which process is defined in this Specification and the Claims as “Interrupting” or to “Interrupt” S/PS/SN nerves.

A thermal energy emitter (e.g., a waveguide an optical fiber, and the like) can be stationed at a desired point in contact with or close to a Target Nerve Tissue, aimed in a desired direction, and thermal energy may be emitted at a desired energy level and for a desired period of time to Interrupt S/PS/SN nerves, alter muscle cells, alter an excessive growth of a Target Nerve Tissue after which this process may be repeated at another point or aimed in another direction. The above process is defined in this Specification and the Claims as “Stationing” or to “Station” a Thermal Energy delivery device.

The thermal energy delivery device may also be Stationed at a desired point in contact with or close to a Target Nerve Tissue, aimed in a desired direction and, while Thermal Energy at a desired level and for a desired period of time is emitted, the thermal energy delivery device may be longitudinally moved back and forth (advanced and withdrawn) at a desired rate of movement over a desired distance for a desired time period, concomitantly or in any desired sequence or order, to apply the thermal energy delivery device longitudinally to the Target Nerve Tissue to, for example, Interrupt S/PS/SN nerves, after which this process may be repeated at another point or aimed in another direction. The above process is defined in this Specification and the Claims as “Moving” or to “Move” a thermal energy delivery device.

A thermal energy delivery device may also be Stationed at a desired point in contact with or close to a Target Nerve Tissue, aimed in a desired direction and, while Thermal Energy at a desired energy level and for a desired period of time is emitted, the thermal energy delivery device may be repetitively rotated, back and forth, laterally over an arc of a desired length, at a desired rate of rotation and for a desired time period, to apply the Thermal Energy radially or latitudinally to the Target Nerve Tissue to Interrupt S/PS/SN nerves, after which this process may be repeated at another point or aimed in another direction. The above process is defined in this Specification and the Claims as “Rotating” or to “Rotate” a thermal energy delivery device.

Also, a thermal energy emitter can be Stationed at a desired point, in contact with or close to a Target Nerve Tissue, aimed in a desired direction and, while Thermal Energy at a desired energy level and for a desired period of time is emitted, the thermal energy delivery device may be Moved and Rotated, concomitantly or in any desired sequence or order, for example, to Interrupt S/PS/SN nerves, which process may then be repeated at another point or aimed in another direction. The above process of Stationing, Moving and Rotating a Source of Thermal Energy is defined in this Specification and the Claims as “Sweeping” or to “Sweep” a thermal energy delivery device.

Any or all of the above processes of Stationing, Moving, Rotating and Sweeping any of the thermal energy delivery device can be separately employed, concomitantly applied or employed in any desired order or sequence.

The thermal energy delivery device, the direction in which it is aimed, the time period of Thermal Energy emission, the distance and rate of movement, the time period thereof, the length of each arc, the rate of rotation and the time period thereof, in Stationing, Moving, Rotating and/or Sweeping are based upon (a) the type, density, color, thermal absorption coefficient and volume of the Target Nerve Tissue to be Denervated or Interrupted, (b) whether denaturation, coagulation or vaporization of the Target Nerve Tissue is desired and (c) the environment or field in which the process is performed, whether in an aqueous field, which cools the Target Nerve Tissue, in a air or CO2 gas field with a spray of sterile water or saline or a cooled gas, preferably a cryogenically cooled gas, to cool the Target Nerve Tissue, in an air or CO2 gas field with no such spray, as well as others, are individually or collectively defined in this Specification and the Claims as the “Environment” in which Interrupting S/PS/SN nerves occurs.

If the Environment does not include the infusion of a sterile, biocompatible irrigating fluid or a spray of such fluid to cool the Target Nerve Tissue, a much lower level of Thermal Energy must be used to avoid charring and damage to the Target Nerve Tissue and adjacent tissues, as will be described later.

### SUMMARY OF INVENTION

An apparatus suitable for denervation, such as renal denervation and the like, comprises an optical fiber situated within a cannula and defining therebetween a channel or confined flow passageway which can deliver a sterile, biocompatible liquid to cool irradiated tissue and to clean debris from distal end position of the optical fiber.

A number of medical conditions can be treated by Interrupting S/PS/SN nerves, denervation of tissues containing S/PS/SN nerves using a thermal energy device, for example, a laser device. These conditions are individually or collectively defined in this Specification and the Claims as “Medical Conditions”.

Also, in this Specification and the Claims, the following terms: (a) to treat, delay progression of or prevent a Medical Condition are individually or collectively defined herein as “Treating” or to “Treat” a Medical Condition; (b) a person suffering from a Medical Condition due to malfunctioning S/PS/SN nerves, and other S/PS/SN disorders of a Target Nerve Tissue of undefined origin is defined herein as a “Patient” (c) delivering Thermal Energy at, onto or into a Target Nerve Tissue is defined herein as “Onto” a Target Nerve Tissue.

A variety of Medical Conditions can be Treated by Stationing, Moving, Rotating and/or Sweeping thermal energy delivery device at or into a Target Nerve Tissue, such methods of delivering Thermal Energy include the following:

(a) A method for Treating a Medical Condition of a Patient comprised of at least one of: Stationing, Moving, Rotating and Sweeping of one of: pulsed laser energy and continuous wave laser energy, delivered Onto a Target Nerve Tissue at an angle of one of: 0°, 0° to 60° and 60° to 90° from optical the axis of the fiber or waveguide one or more needles, each containing an optical fiber for delivery of laser energy to a desired depth within a Target Nerve Tissue, and multiple beams of at least one of: laser, x-ray, proton, RF, MW and US energy, focused to intersect at a desired point, to Interrupt S/PS/SN nerves of the renal arteries of the kidneys, which contain an aggregation of S/PS/SN nerves, the Medical Condition of the Patient being at least one of: hypertension (systolic blood pressure equal to or greater than 140 mm of mercury), chronic renal failure, acute renal failure, end stage renal disease, left ventricular hypertrophy, chronic heart failure, acute heart failure, acute myocardial infarction (heart attack), stroke, insulin resistance, Type II diabetes mellitus, sleep apnea and other S/PS/SN nerve-affected, kidney-related disorders;

(b) A method for Treating a Medical Condition of a Patient comprised of at least one of: Stationing, Moving, Rotating and Sweeping of one of: pulsed laser energy and continuous wave laser energy, delivered Onto a Target Nerve Tissue at an angle of at least one of: 0°, 0° to 60° and 60° to 90° from the axis of the thermal energy emitter, including one or more needles, each containing an optical fiber for delivery of laser energy to a desired depth within a Target Nerve Tissue, and multiple beams of at least one of: laser, x-ray, proton, RF, MW or US energy, focused to intersect at a desired point, to at least one of: Interrupt S/PS/SN nerves, including the lower trachea and branches of the bronchi, as well as the vagus nerve of the bronchi of the lungs, which contain an aggregation of S/PS/SN nerves, the Medical Condition of the Patient being at least one of: asthma, chronic obstructive pulmonary disease and other S/PS/SN nerve and muscle cell-affected, pulmonary-related disorders;

(c) A method for Treating a Medical Condition of a Patient comprised of at least one of: Stationing, Moving, Rotating and Sweeping thermal energy delivery device Onto a Target Nerve Tissue to Interrupt S/PS/SN nerves, including at least one of: the vagus and splanchnic nerves, within at least one of: (i) the esophagus, as well as the esophageal artery and vein of the esophagus; (ii) the hepatic artery and veins of the liver; (iii) the pancreas, as well as the pancreatic and pancreaticoduodenal arteries and their respective veins of the pancreas; (iv) the stomach, as well as the abdominal, celiac and gastroepiploic arteries and their respective veins of the stomach; (v) the duodenum, as well as the celiac, gastroduodenal and pancreaticoduodenal arteries, their respective veins, and the portal vein of the duodenum; and (vi) the intestines, as well as the mesenteric, colic and sigmoid arteries and their respective veins of the intestines, each of the above containing an aggregation of S/PS/SN nerves, the Medical Condition of the Patient being at least one of: Type II diabetes mellitus, insulin resistance, atherosclerosis (by changing the manner in which fat from the diet and fat released from stored fat is metabolized), obesity (by at least one of: affecting the sensation of satiety, the manner in which fat from the diet and fat released from stored fat is metabolized and the means by which the volume of fat stored is controlled), ulcers, irritable bowel syndrome, celiac disease and other S/PS/SN nerve-affected, digestive system-related disorders; (d) A method for Treating a Medical Condition of a Patient comprised of at least one of:

Stationing, Moving, Rotating and Sweeping thermal energy delivery deviceOnto a Target Nerve Tissue to Interrupt S/PS/SN nerves, including at least one of: the vagus, carotid and cervical cardiac nerves, of at least one of: the major veins of the heart and at least one of the fat pads of the heart, preferably the last 50 mm of the right pulmonary veins at their junction with the left atrium of the heart and at least three of the fat pads of the heart located (i) on the posteroinferior surface of the heart beneath the left atrium, (ii) on the posteroinferior surface of the heart beneath the right atrium and (iii) on the anterior inferior surface of the heart beneath the left and right atria, each of the above containing an aggregation of S/PS/SN nerves, the Medical Condition of the Patient being at least one of: arrhythmia, paroxysmal arrhythmia, bradyarrythmia, bradycardia, reduced myocardial contractibility, atrial fibrillation, ventricular fibrillation, cardiac arrest, chronic heart failure, acute heart failure, acute myocardial infarction, stroke, sleep apnea and other S/PS/SN nerve-affected, cardiac-related disorders;

(e) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the hips, legs and feet, as well as at least one of: the vagus and splanchnic nerves, of at least one of: the popliteal, tibial, ilium, sacral and peroneal arteries and their respective veins and the saphenous vein of at least one of: the hip, legs and feet, each of the above containing an aggregation of S/PS/SN nerves, the Medical Condition of the Patient being at least one of: peripheral neuropathy, chronic leg cramps, claudication, edema, amputation, restless leg syndrome and other S/PS/SN nerve-affected, peripheral (below the waist) artery-related disorders;

(f) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the brain, as well as at least one of: the carotid, vagus and cervical nerves, of at least one of: the carotid and vertebral arteries and their respective veins of the brain, each of the above containing an aggregation of S/PS/SN nerves, the Medical Condition of the Patient being at least one of: epilepsy, seizures, convulsions, fragile X syndrome, autism, multiple sclerosis, amyotrophic lateral sclerosis (ALS), myasthemia gravis, severe depression, migraine headaches, schizophrenia, psychosis, anxiety, Parkinson's disease, Huntington's disease, senile dementia, Alzheimer's disease and other S/PS/SN nerve-affected, central nervous system-related disorders;

(g) A method for Treating a Medical Condition of a Patient comprised of at least one of: Stationing, Moving and Sweeping a thermal energy delivery device Onto a Target Nerve Tissue to Interrupt S/PS/SN nerves, including the thalamus, as well as at least one of: the carotid, vagus and cervical nerves, of at least one of: the basilar, carotid, cerebral and caudate arteries and their respective veins and the thalomostrate vein of the thalamus, each of the above containing an aggregation of S/PS/SN nerves, the Medical Condition of the Patient being at least one of: insomnia, narcolepsy, multiple sclerosis, amyotrophic lateral sclerosis (ALS), myasthenia gravis, the need to at least one of: repair tissue and attack at least one of bacteria, a virus or cancer cells (by affecting the means by which the volume, rate of maturism and release of white cells and stem cells is controlled) and other S/PS/SN nerve-affected, thalamus-related disorders;

(h) A method of Treating a Medical Condition of a Patient comprised of at least one of: Stationing, Moving, Rotating and Sweeping a thermal energy delivery device Onto a Target Nerve Tissue to Interrupt S/PS/SN nerves, including hypothalamus and pituitary gland, as well as at least one of: the cervical, carotid and vagus nerves, of at least one of: the basilar, cerebral, hypothalamic, carotid and caudate arteries and their respective veins of the hypothalamus and the pituitary and hypophyseal arteries and their respective veins of the pituitary gland, each of the above containing an aggregation of S/PS/SN nerves, the Medical Condition of the Patient being at least one of: hypogonadism, hypothyroxinemia (low concentration of thryroxine in the blood stream), hyperthyroidism (high concentration of thyroxin in the blood stream), infertility, irregular menorrhagia, Type II diabetes mellitus and other S/PS/SN nerve-affected hypothalamus and pituitary gland-related disorders;

(i) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the following joints, as well as at least one of: the vagus, sinu-vertebral, cervical, spinous and sciatic nerves of the vertebra, the dorsal primary ramus nerves of the facets and/or their branches, and the sinu-vertebral nerves of the sacrum, and their branches in the joints of at least the two vertebra above the sacrum, of at least one of: (i) the vertebra, the joint of the sacrum and ilium, the hips, long bones of the arms and legs and the small bones of the ankles, feet, toes, wrists, hands and fingers; (ii) the vertebral, spinal and intercostal arteries, their respective veins and the vena cava vein of the vertebra; (iii) the sacral artery and vein of the sacrum; (iv) the subclavian, brachial, humeral and scapular arteries and their respective veins of the joints of the shoulders; (v) the radial and ulnar arteries and their respective veins of the joints of the elbows; (vi) the palmar and digital arteries and their respective veins of the joints of at least one of: the wrists, hands and fingers; (vii) the femoral artery and vein and saphenous vein of the joints of the hips; (viii) the femoral, ilium and popliteal arteries and their respective veins and the saphenous vein of the joints of the knees; and (ix) the popliteal, tibial and peroneal arteries and their respective veins and the saphenous vein of the joints of at least one of: the ankles, feet and toes, each of the above containing an aggregation of S/PS nerves, the Medical Condition of the Patient being pain arising from at least one of: arthritis, avascular necrosis, osteonecrosis, osteocarcoma and physical damage of at least one of: the vertebra, spinal discs and bones, and pain arising from the joints of at least one of: the facets, sacrum and ilium, shoulders, elbows, wrists, hands, fingers, hips, knees, ankles, feet and toes and other S/PS/SN nerve-affected, vertebral, spinal disc, bone and joint-related disorders.

(j) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the spleen, as well as at least one of: the vagus and splanchinic nerves of at least one of: the splenic arteries and veins of the spleen, each of the above containing an aggregation of S/PS/SN nerves, the Medical Condition of the Patient being one of: hypohemoglobia (anemia) and hyperhemoglobia (by affecting the means by which the volume and rate of destruction of red cells is controlled) and other S/PS/SN nerve-affected, spleen-related blood disorders;

(k) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the uterus, as well as at least one of: the vagus and splanchnic nerves of at least one of: the uterine arteries and veins of the uterus, each of which contain an aggregation of S/PS/SN nerves, the Medical Condition of the Patient being at least one of: uterine cramps, hot flashes, amenorrhea, dysmenorrhea and infertility (to affect the means by which the volume, type and rate of production of reproductive hormones are controlled) and other S/PS/SN nerve-affected, uterine-related disorders;

(l) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the ovaries, as well as at least one of: the vagus and splanchnic nerves of at least one of: the ilium, uterine and ovarian arteries and their respective veins of the ovaries, each of the above containing an aggregation of S/PS/SN nerves, the Medical Condition of the Patient being infertility (by affecting the means by which the maturity of eggs, the number of eggs released and their rate of release is controlled), menopause (by affecting the means by which the type, volume and rate of production of reproductive hormones is controlled) and other S/PS/SN nerve-affected, ovarian-related disorders;

(m) A method for Treating a Medical Condition of a Patient comprised of at least one of: Stationing, Moving, Rotating and Sweeping a thermal energy delivery device Onto a Target Nerve Tissue to Interrupt S/PS/SN nerves, including the testes, as well as at least one of: the pelvic and splanchnic nerves of at least one of: the ilium, epigastric and testicular arteries and their respective veins of the testes, each of the above containing an aggregation of S/PS/SN nerves, the Medical Condition of the Patient being at least one of: aspermia (male infertility, by affecting the means by which the volume and maturity of sperm produced and released is controlled) and hypogonadism (by affecting the means by which the volume and rate of production of testosterone and its release is controlled) and other S/PS/SN nerve-affected, testis-related disorders;

(n) A method of Treating a Medical Condition of a Patient comprised of at least one of: Stationing, Moving, Rotating and Sweeping a thermal energy delivery device Onto a Target Nerve Tissue to Interrupt S/PS/SN nerves, including the penis, as well as at least one of: the pelvic and splanchnic nerves of at least one of: the pudental arteries and veins of the penis, each of the above containing an aggregation of S/PS/SN nerves, the Medical Condition of the Patient being at least one of male impotence (by affecting the means by which blood flow to cause erection of the penis is controlled and maintained) and other S/PS/SN nerve-affected, penile-related disorders;

(o) A method of treating a Medical Condition of a Patient comprised of at least one of: Stationing, Moving, Rotating and Sweeping a thermal energy delivery device Onto a Target Nerve Tissue to Interrupt S/PS/SN nerves, including the rectum or anus, as well as at least one of: the pelvic and splanchnic nerves of at least one of: the rectal artery and vein of the rectum and anus, the Medical Condition of the Patient being fecal incontinence and other S/PS/SN nerve-affected, rectal and anal-related conditions;

(p) A method of Treating a Medical Condition of a Patient comprised of at least one of: Stationing, Moving, Rotating and Sweeping a thermal energy delivery device Onto a Target Nerve Tissue to Interrupt S/PS/SN nerves including the scalp, as well as of at least one of: the frontal and parietal branches of the superficial arteries and veins of the scalp, each of which contain an aggregation of S/PS/SN nerves, the Medical Condition of the Patient being at least one of: alopecia (baldness, by affecting the means by which at least one of: the type and volume of testosterone is produced and its rate of release is controlled, and the means by which the rate of flow and volume of blood delivered to the dermis of the scalp is controlled) and dandruff (by affecting the means by which the volume of blood delivered to the scalp and its rate of flow is controlled) and other S/PS/SN nerve-affected, scalp-related disorders; and

(q) A method for Treating a Medical Condition of a Patient comprised of at least one of: Stationing, Moving, Rotating and Sweeping a thermal energy delivery device Onto a Target Nerve Tissue to at least one of: Interrupt the S/PS/SN nerve endings in the capsule of the facet joints of the vertebra; the medial branches of the dorsal primary ramus nerve of the facet joints of the vertebra; and the vertebral, sinu-vertebral, vagus and splanchnic nerves of the sacrum and ilium of the hip, preferably the sinu-vertebral nerves of the sacrum and their branches into at least the two vertebra above the sacrum, the Medical Condition of the Patient being at least one of neck, shoulder, back, hip and leg pain originating in the facets of the vertebra and joint of the sacrum and ilium and other S/PS/SN nerve-affected, facet and sacroiliac joint-related disorders.

Pulsed laser energy or other thermal energy delivery device may be introduced into an artery, vein, bronchi, hollow or solid organ, gland or duct, in a percutaneous, intra-luminal procedure through a conventional guiding catheter, cannula, endoscope, bronchoscope or a surgically created passageway to Interrupt S/PS/SN nerves of the artery, vein, bronchi, hollow or solid organ, gland or duct from inside the artery, vein, bronchi, hollow or solid organ, gland or duct to Treat a Medical Condition of a Patient.

Pulsed laser energy or other thermal energy delivery device may also be introduced through an endoscope, laparoscope, or surgically created passageway, which may be cannulated, to Interrupt S/PS/SN nerves of an artery, vein, bronchi, hollow or solid organ, gland or duct from outside the artery, vein, bronchi, hollow or solid organ, gland or duct in an endoscopic or laparoscopic procedure or through a surgically created passageway, which may be cannulated, to Treat a Medical Condition of a Patient.

When “Rotated” is used herein, it means repetitive rotations of the thermal energy beam from its starting point to its end point and back, during the selected rotation time period, such as 0.5 to 2 cycles per second, preferably about one cycle per second, so the operator can time each cycle by mentally counting “one thousand”, “two thousand”, etc.

Target Nerve Tissues can be altered by Stationing, Moving, Rotating and/or Sweeping a beam of Thermal Energy, in any desired sequence or combination, Onto a Target Nerve Tissue. One of the preferred thermal energy delivery deviceis laser energy, preferably pulsed laser energy, most preferably pulsed CTH:YAG or Holmium laser energy, transmitted through a 0° straight-ahead firing, a 0° to 60° angled firing or a 60° to 90° side firing delivery device, as described below.

In the first side firing embodiment of a suitable of laser energy delivery device, the proximal end of a conventional, end-firing optical fiber is optically coupled to a source of laser energy and a metal tip for diverting laser energy laterally from the axis of the optical fiber is fixedly attached by crimping and/or an adhesive to the distal end of the optical fiber. The metal tip is preferably made entirely of or coated with a material highly reflective to the wavelength of laser energy being used, such as silver or gold, stainless steel which has been plated with silver or gold, with a thickness of preferably at least five preferably more thousandths of an inch, stainless steel with an insert of gold or silver, preferably with a thickness of ten to twenty or more thousandths of an inch, or stainless steel coated with a dielectric.

Preferably, the protective buffer coating and any polymer cladding are removed from the distal end portion of the optical fiber prior to attachment of the metal tip. Alternatively, the metal tip can also be attached by an adhesive and/or crimping to the protective buffer coating covering the optical fiber, if desired.

The metal tip defines a central cavity, into which the distal end of the optical fiber extends. The distal end surface of the cavity is inclined at an angle of about 35° to 50°, preferably at an angle of about 45° from the optical axis of the optical fiber. The open portion of the cavity allows laser energy, reflected by the inclined, reflective metal surface, to be emitted from the cavity in the metal tip at an angle of about 90° from the axis of the optical fiber, in accordance with Snell's Law.

For ease of manufacture and durability, the entire metal tip is preferably made of a highly reflective material, such as very pure gold or silver, both of which are malleable, preferably silver, which has about the same reflectivity as gold, but is much less expensive. Most preferably, the silver should be about 95.5% pure.

In the side firing device described above, the optical fiber extends from the source of laser energy, through a passageway or channel, which extends lengthwise through a metal or rigid plastic handpiece, for ease of use.

The optical fiber extends through the passageway and is fixedly attached within the proximal end of the handpiece by an adhesive or the like, which serves to sealingly close the proximal end of the passageway in the handpiece. Alternatively the optical fiber may be removably attached within the proximal end of the handpiece by a compression fitting, as known in the art, which sealingly closes the proximal end of the handpiece.

In addition to sealingly closing the distal end of the handpiece, a compression fitting, when loosened, enables the side firing device to be removed, cleaned and resterilized for use in another procedure, and permits the handpiece to be cleaned, resterilized and used again, or vice versa. The optical fiber extends distally from the handpiece a desired distance, with its distal end modified to emit laser energy laterally from the axis of the optical fiber, as described above.

Optionally, the optical fiber of the side firing device can extend through a plastic cannula extruded with a central or eccentric channel for the optical fiber and one or more surrounding channels for other purposes. The plastic cannula can be made of a flexible, semi-flexible, semi-rigid or rigid biocompatible plastic, preferably a very flexible plastic.

The proximal end of the plastic cannula may be fixedly attached by an adhesive or other means within (a) the distal end of the connector of the optical fiber at or near the laser, (b) at least about 6 cm proximal from the proximal end of the handpiece or (c) within the distal end of the handpiece.

The distal end of the multi-channel cannula can extend (a) up to the proximal end of the crimped portion of the metal tip, (b) over the crimped portion of the metal tip, (c) over the crimped portion and over the metal tip, up to the area of laser energy emission or (d) over the crimped portion and up to the distal end of the metal tip, with a port for emission of laser energy over the 45° inclined surface of the metal tip. For example, the optional, multi-channel plastic cannula can be extruded with a central channel for the optical fiber to center the side firing device within a blood vessel, bronchi, hollow organ or duct, or with an eccentric channel for the optical fiber to position the side firing device close to the wall of the blood vessel, bronchi, hollow organ or duct.

The central or eccentric channel for the optical fiber can have, for example, three surrounding longitudinal channels, one channel for infusion of a sterile, biocompatible irrigation fluid, such as saline or water, to cool the laser energy emitting surface of the side firing device and the Target Nerve Tissue, one channel for infusion of a sterile, biocompatible fluid, such as saline or water, to inflate a concentric or eccentric balloon, which may be mounted on the exterior of the multi-channel cannula proximal to the proximal end of the metal tip, to either center the side firing device in a blood vessel, bronchi, duct or hollow organ, or to position the laser energy emitting surface of the side firing device close to the wall of the blood vessel, bronchi, duct or hollow organ, and one channel to enable the sterile, biocompatible fluid to return from the balloon and flow into a drain or collection bottle.

If the side firing device is used in a bronchi of the lung, a sterile, biocompatible liquid, such as saline, water, CO2 gas or nitrogen, can be used to inflate the balloon. If the side firing device is used inside a blood vessel, inflating the balloon prevents blood from traveling beyond the balloon, and the infusion of a biocompatible fluid, such as sterile water or saline to cool the laser energy emitting surface of the side firing device and the surface of the Target Nerve Tissue, also forces blood away from the laser energy emitting surface of the side firing device, avoiding the coagulation of blood, which could cause a blood clot in the lung, brain or elsewhere, potentially a life-threatening condition.

If the plastic cannula is extruded with only two surrounding channels, one for infusion of an irrigation fluid to cool the side firing surface of the device and the Target Nerve Tissue and one to inflate the balloon, and the side firing device is to be used in a blood vessel, it is necessary to purge the air from the channels used to (a) infuse an irrigating fluid and (b) inflate the balloon, which could, if air is left in place in the channels and expelled into a blood vessel, cause an air embolism, a life threatening condition.

In the cannula described above with only two surrounding channels, the balloon can be manufactured with one or more holes or vents to allow the air to escape into the atmosphere when the channel in the cannula to inflate the balloon is filled with a fluid. Before being inserted into a patient, the sterile, biocompatible fluid is infused into both of the surrounding channels, until liquid is seen to escape the holes or vents in the balloon and the irrigation channel, indicating that purging of air from all the channels has been completed.

If the cannula is extruded with three surrounding channels, and is to be used with the above described balloon with very small holes to allow air to be expelled from the balloon, when the balloon inflation channel is filled with a sterile, biocompatible fluid, the third channel can be plugged with an adhesive or the like at its proximal and distal ends, and not used.

As mentioned above, the balloon can be eccentric, preferably wider on the side opposite the side from which laser energy emitted, and narrower on the side from which laser energy is emitted, to position the laser energy emitting side of the side firing device close to the wall of the blood vessel, duct, bronchi or hollow organ.

Alternatively, the balloon may be mounted on the side of the plastic cannula opposite the side from which laser energy is emitted, to force the side firing device against the wall of the blood vessel, duct, bronchi or hollow organ, while preventing blood flow, as described above.

A luer fitting, as known in the art, may be sealingly and fixedly attached within and extends through the body of the handpiece and is in fluid communication with the central passageway in the handpiece and the irrigation fluid channel of the multi-channel cannula. If a balloon is to be inflated, a separate luer fitting can be attached to the plastic cannula in fluid communication with the channel in the plastic cannula for inflation of the balloon, and a third luer fitting can be attached to the cannula in fluid communication with the fluid return channel, to enable the returned fluid to flow through a discharge tube into a drain or collection bottle.

Alternatively, all luer fittings can be attached to the multi-channel cannula, each in fluid communication with one channel of the multi-channel cannula, at one point or points at least about 6 cm proximal to the proximal end of the handpiece, so the luer fittings and the attached fluid lines do not interfere with the surgeon's use of the handpiece to Station, Move, Rotate and Sweep the distal, side firing portion of the device.

To provide support for the luer fittings at their junction with each of their respective channels of the hollow, multi-channel cannula, a rigid plastic or metal collar can be adhesively attached to the multi-channel cannula and the luer fitting fluid lines. All three luer fittings may be attached with a radial collar, a collar extending longitudinally along the exterior of the multi-channel cannula, or a separate collar for each luer fitting may be employed.

If the multi-channel cannula extends over the laser energy emitting portion of the side firing device, the multi-lumen cannula must have a port for emission of laser energy opposite the surface from which laser energy is emitted.

In the second embodiment of the present invention, an optical fiber, optically coupled to a source of laser energy, and from whose distal end portion the plastic buffer coating and any polymer cladding has been removed is utilized. The optical fiber extends through a hollow passageway extending lengthwise through the body of a handpiece. The optical fiber having a “barrel” distal end portion is fixedly attached to the handpiece, preferably within the proximal end of the handpiece, in a manner which sealingly closes the proximal end of the passageway, or allows the optical fiber to be sealingly and removeably attached in the proximal end of the handpiece, as described above.

The barrel distal end of the optical fiber is beveled at an angle of about 35° to 45°, preferably at an angle of about 40° to 41°, for optimal reflection and laser energy transmission efficiency. A distally closed-ended capillary tube is disposed over and fixedly and sealingly attached by an adhesive, thermal fusing, a combination of the foregoing or other means known in the art, to the bared distal end portion of the optical fiber. Fixedly and sealingly disposing a closed-ended capillary tube over the distal end of the optical fiber creates an air environment opposite the beveled, distal end surface of the optical fiber.

The difference in the refractive index of air, versus the refractive index of the quartz or fused silica core of the optical fiber, enables total internal reflection of the light energy to occur laterally at an angle of double the bevel angle, according to Snell's Law. If the distal end of the optical fiber is beveled at an angle of 40° to 41°, laser energy is emitted at an angle of about 80° to 82° out of a side laser energy emission and fat entry port near the distal end of the hollow liposuction cannula opposite the beveled, distal end surface of the optical fiber.

Likewise, the optional plastic cannula described above, the optional balloon configurations described above, the use of the channels described above, and the luer fittings communicating with each of the surrounding channels of the multi-channel cannula, as described above, can be used with this second embodiment of the side firing device. Alternatively, the plastic cannula can extend over the side firing device, with a port for emission of laser energy positioned in the path of laser energy emission, as described above.

In the third embodiment of the device of the present invention, if a laser generating energy at wavelengths of about 1400 to 1500 nanometers (nm) or 1800 to 3000 mm, which wavelengths of light are highly absorbed by water, is emitted through an optical fiber, whose distal end has been beveled at an angle of 35° to 45°, preferably at an angle of about 40° to 41° for optimal reflection and laser energy transmission efficiency, in an aqueous liquid environment, the closed-ended capillary tube can be eliminated. The first portion of the laser energy emitted vaporizes a portion of the aqueous irrigation liquid infused through an endoscope, or a channel in the optional multi-channel plastic cannula described above and creates a steam bubble opposite the beveled, distal end surface of the optical fiber.

The steam bubble has an index of refraction sufficiently lower than that of the refractive index of the quartz or fused silica core of the optical fiber to cause the laser energy to be internally reflected, according to Snell's Law, at an angle of about 80° to 82° out of the side port in the cannula, as described above. However, the laser energy emitting surface of this embodiment must be positioned close to but not in contact with the Target Nerve Tissue, or much of the laser energy will be wasted vaporizing any intervening aqueous irrigation liquid. Contacting the Target Nerve Tissue can cause tissue to adhere to the laser energy emitting surface of the side firing device, reducing its transmission efficiency.

Likewise, the optional multi-channel cannula described above, the optional balloon configurations described above, the surrounding channels described above, optionally extending the multi-channel cannula over the side firing device, with a port for emission of laser energy, as described above, and the luer fittings in fluid communication with each of the surrounding channels of the cannula, can be used with this embodiment of the side firing device.

For use in surgically created passageways, or in endoscopic or laparoscopic procedures, an aiming beam of a desired color, for example, red or green, such as from a helium neon (HeNe), a diode laser or other laser delivering about 1 to 5 milliwatts, as known in the art, can be transmitted through the optical fiber and reflected at about the same angle as the therapeutic laser energy, which may be of an invisible wavelength, to enable the operator to see the direction in which the therapeutic laser energy is being emitted. Green may be preferred, as red may be more difficult to discern in a region containing blood.

Laser energy at wavelengths of about 300 to 400 nm must be used through optical fibers with a hydroxyl ion content of 600 to 800 ppm, called high-OH fibers, to prevent excessive loss of laser energy at these wavelengths. Laser energy at wavelengths of about 400 to 1400 nm and about 1500 to 1800 nm can be used through conventional optical fibers with a hydroxyl ion content of 100 to 600 ppm or, preferably, for more efficient transmission efficiency, through or optical fibers with a low hydroxyl ion content, of about 0.1 to 100 ppm, to reduce transmission losses.

An optical fiber with a low hydroxyl ion content of about 1 to 100 parts per million (“ppm”), called a low-OH fiber, should be used with lasers whose wavelength is 1400 to 1500 or 1800 to 2300 nm to prevent excessive loss of laser energy. An optical fiber with an extremely low hydroxyl ion content of about 0.01 to 1 ppm, called an ultra low-OH fiber, should be used with lasers emitting energy at a wavelength of 2300 to 3000 nm, to avoid excessive loss of laser energy at these wavelengths.

Laser energy at wavelengths of about 1400 to 1500 nm and about 1800 to 3000 nm cannot be effectively used through the first embodiment of the present invention described above, in which an optical fiber whose distal end is opposed to an inclined surface of an attached reflective metal tip, as described above, as too much of the laser energy will be lost vaporizing the intervening saline or water infused to cool the laser energy emitting surface of the side firing device and the Target Nerve Tissue.

Likewise laser energy of wavelengths of 300 to 1400 nm and 1500 to 1800 nm cannot be effectively used through the third embodiment of the present invention, in which the distal end of the optical fiber is beveled at an angle of 40° to 41°, without being fixedly encased within a closed-ended capillary tube, as these wavelengths of laser energy are not highly absorbed by water and will not create a stream bubble with a refractive index opposite the beveled surface of the optical fiber necessary for total internal reflection of the laser energy.

However, contrary to common wisdom in the laser field, we discovered that all wavelengths of laser energy from about 300 nm to 3000 nm, used through optical fibers with hydroxyl ion contents applicable to each, as described above, can be used through the second embodiment of the present invention, namely the side firing device described above, in which the distal end of the optical fiber is beveled at an angle of about 35° to 45°, most preferably at an angle of about 40° to 41°, and is fixedly and sealingly encased by a distally closed-ended capillary tube to create the air environment needed for total internal reflection of laser energy to occur.

A variety of lasers fall within wavelengths of about 300 nm to 3000 nm. For example, lasers emitting at 300 to 400 nm, include, for example, excited dimer lasers, called “excimer” or “exciplex” lasers, including Xenon Chloride (XeCl) lasers emitting at a wavelength of about 308 nm and Xenon Fluoride (XeFl) lasers emitting at a wavelength of about 351 nm, which wavelengths are highly absorbed by molecular bonds, causing disruption and vaporization of tissue. However, the light extinction depth of excimer laser energy is only about 5 microns, excimer lasers are generally limited to powers of only about 10 watts, and they use highly toxic gasses, which can be dangerous in a medical facility.

Lasers emitting at 400 nm to 1400 nm and from 1500 nm to 1800 nm include, for example, an argon laser emitting at about 488 to 514 nm, a KTP laser emitting at a wavelength of 532 nm, which is highly absorbed by a red pigment, such as oxygenated hemoglobin in blood, a diode laser emitting at wavelengths of about 600 nm to 1400 nm, an alexandrite laser emitting at a wavelength of 810 nm, and a Nd:YAG laser emitting at a wavelength of 1064 nm, which wavelengths are absorbed to a modest extent by pigments and to a limited extent in water. These lasers have light extinction depths ranging from 800 to 4000 microns.

Lasers emitting at 1400 to 1500 nm and from 1800 to 3000 nm include, for example, a certain diode laser emitting at a wavelength of about 1470 nm, a Thulium:YAG laser emitting pulsed or continuous wave laser energy at a wavelength of about 2000 nm, a Chromium, Thulium, Holmium or CTH:YAG laser, commonly referred to as a “Holmium laser”, emitting pulsed laser energy at a wavelength of about 2100 nm, a YSGG:YAG laser emitting pulsed laser energy at a wavelength of about 2106 nm, the light extinction depth of the CTH:YAG and YSGG:YAG lasers in tissue is about 400 microns, and an Erbium:YAG laser emitting pulsed laser energy at a wavelength of about 2900 nm, whose light extinction depth in tissue is only about 50 microns, all of which wavelengths are highly absorbed by water, a constituent of all tissues, as well as the irrigation liquids commonly used in endoscopic procedures.

While all of the above-described wavelengths of laser energy can be used through the second embodiment of the side firing device of the present invention, provided the core of the optical fiber has a sufficiently low hydroxyl-ion content of an appropriate amount for effective transmission of each laser's wavelength, pulsed Holmium laser energy is preferred, as its depth of penetration in tissue is ideal for use in arteries, veins, bronchi and other Target Nerve Tissues with a wall thickness of about 1 to 2 mm. And, very short, about 350 microsecond, pulses of laser energy, leave time for the tissue to cool between pulses of laser energy.

If the wall thickness of a Target Nerve Tissue is larger than 1 to 2 mm, (a) a longer emission time may be used to enable thermal diffusion of the laser energy to occur, (b) a laser whose wavelength penetrates tissue to a greater depth can be employed, (c) multiple beams of laser or other Thermal Energy may converge at a desired point within the Target Nerve Tissue, or (d) one or more needles, each containing an optical fiber, may be inserted to deliver laser energy at a desired depth within the Target Nerve Tissue.

The handpiece can have a raised button, preferably in a contrasting color, i.e., color may be significantly different from that of the handpiece, which the operator can see and sense by tactile feel. The button can be positioned on the side of the handpiece from which the laser energy is emitted or, preferably, on the side of the handpiece opposite the side from which the laser energy is emitted. If so positioned, when the handpiece is gripped, the forefinger or thumb of the operator, touching the button, points in the direction in which the laser energy will be emitted.

In a preferred version of the second embodiment of the present invention, the beveled, distal end surface of the optical fiber may be encased within a closed-ended capillary tube with a substantially thinner wall thickness, which causes the laser energy to be more widely diverged, enabling a greater volume of Target Nerve Tissue to be altered and allows the side firing device to be rotated through an arc of only about 90° to achieve the same effect. In this embodiment, the wall thickness of the capillary tube is not greater than 350 microns, compared to a typical wall thickness of about 500 microns of the capillary tube in the second embodiment of the side firing devices described above.

Today, all side firing devices are made with optical fibers with a core diameter of about 500 to 600 microns or larger, as conventional wisdom in the medical laser field is such core diameters are necessary to efficiently capture and transmit 100 or more watts of laser energy. Such diameters of optical fibers are too inflexible to be used through a conventional guiding catheter to access, for example, to the main renal arteries from the aorta through a bend of about 90°, or through a flexible endoscope, which may be bent (articulated) at an angle of 90° or more to access a Target Nerve Tissue, for example, to deliver a thermal energy delivery device to the outer surface of the front, top, back and bottom of an artery, vein, bronchi, duct, gland vertebra, bone, organ or other Target Nerve Tissue.

Contrary to common wisdom, we tested optical fibers with core diameters successively less than 550 microns and discovered that Holmium and other wavelengths of laser energy can efficiently deliver 100 watts of power through optical fibers as small as 365 microns or even smaller. In the process of constructing and testing optical fibers with a core diameter of 365 microns, we created the smallest side firing devices ever made, with an O.D. as small as 1.5 mm (conventional side firing devices are usually 2 mm to 2.3 mm in O.D. or larger.

These smaller diameter core fibers enable side firing devices to be used through a metal cannula with a bend near its distal end, a conventional guiding catheter or a rigid endoscope, whose distal end may be flexible and bent or articulated by wires or other means, by up to 90° or more, provided the bend radius is not smaller than 1 to 1.5 cm, as described below, which could cause laser energy to leak at the bend and damage the cannula, guiding catheter or flexible endoscope.

Another improvement we conceived is the use of a heat shrinkable plastic tube, which is shrunk over the distal end portion of the optical fiber, the junction of the optical fiber with the proximal end of the metal tip or capillary tube, over the proximal end portion of the capillary tube and terminates just proximal to the area of laser energy emission from the capillary tube. The heat shrunk tube reduces the risk of the capillary tube from being dislodged from the optical fiber, as a safety measure. An adhesive may also be applied to the area to be covered by the heat shrinkable tube, prior to the heat shrinking process, as an additional safety measure.

The unique construction of any of the side firing devices described above permits effective and uniform Interrupt ion of S/PS/SN nerves. These side firing devices can be used in one or more novel methods of use to achieve a significantly more effective, safe and uniform Interruption of S/PS/SN nerves and tissues containing S/PS/SN nerves.

After Stationing a side firing device, or other thermal energy delivery device, in a blood vessel, bronchi, duct, or hollow organ, or in a solid Target Nerve Tissue or surgically created passageway, the button on the handpiece can be positioned, for example, at 3 o'clock, causing Thermal Energy to be emitted at 9 o'clock, after which the button can be successively positioned at 6, 9 and 12 o'clock, causing Thermal Energy to be emitted at 12, 3 and 6 o'clock. This process can be started at any of such positions.

Thermal Energy can be emitted, for example, at an energy level of about 3 to 40 watts, preferably about 5 to 20 watts, preferably about 8 to 15 watts, provided the Environment is an aqueous liquid, which cools the Target Nerve Tissue.

If the Environment is air or a gas, such as CO2 gas, the emission of such amount of Thermal Energy is concomitantly accompanied by the infusion of a sterile, biocompatible irrigating liquid or a spray of a sterile, biocompatible irrigation fluid, preferably sterile water or saline or a cold or cryogenically cooled biocompatible gas, such as CO2 or nitrogen, to cool the Target Nerve Tissue.

However, if no means to concomitantly cool the Target Nerve Tissue is used, lower laser energy must be applied, for example, at a power level of about 0.05 to 3 watts, preferably at about 0.1 to 1.5 watts, to avoid a build-up of heat that could damage the Target Nerve Tissue or adjacent tissue. The level of laser energy power and its duration is dependent on the area and volume of Target Nerve Tissue to be Interrupted, the pulse repletion rate and the rate of Moving, Rotating and/or Sweeping the Source of Thermal Energy and the time period thereof, and is determined by the physician performing the procedure.

If the side firing device or other thermal energy delivery device is Moved, each longitudinal movement can be for about 0.5 to 3 seconds, preferably about 1 to 2 seconds, in each direction, depending upon the distance the side firing device is to be extended from the distal end of a guiding catheter, cannula, endoscope, laparoscope or though a surgically created passageway, as determined by the physician performing the procedure.

If the side firing device or other thermal energy delivery device is Rotated through an arc of about 90° to 120° while Stationed and/or Moved, its rate of rotation is preferably about one arc per second, for the reasons described above.

To Treat certain Medical Conditions of a Patient, it may be difficult, impossible or impractical to use a side firing device. In such instances, a straight optical fiber may be inserted through a rigid endoscope or laparoscope, or through a rigid endoscope or laparoscope whose distal end portion, about 5 to 10 cm in length, may be articulated or bent at an angle up to 90° or greater, usually by manipulating wires contained in the distally flexible device, as known in the art, provided the bend radius is not smaller than 1 to 1.5 cm, as described below. The above described scopes can be Stationed, Moved, Rotated and/or Swept, individually or in any desired combination or sequence, to Treat the Medical Condition from outside the blood vessel, bronchi, duct, hollow or solid organ, growth of excessive tissue or other Target Nerve Tissue.

Also, in the treatment of certain Medical Conditions, an optical fiber may extend from a source of laser energy through a handpiece and a prior art, hollow metal or rigid plastic cannula, preferably made of medical grade stainless steel. The proximal end of the optical fiber is fixedly attached within the distal end of the handpiece by an adhesive, as known in the art.

The cannula's distal end portion can be straight or bent to form an angle in the range, for example, of 10°, 20°, 30°, 40°, 50° or 60° from the longitudinal axis, or any other desired angle, providing the bend radius does not exceed 1 to 1.5 cm, as described below. Such a cannula and the optical fiber extending therethrough, may be inserted through a body orifice or a surgically created passageway and used under direct viewing, (a) through a rigid endoscope or laparoscope, (b) through an endoscope or laparoscope with a distal, flexible portion, or (c) by ultrasound, fluoroscopic or x-ray imaging. The optical fiber device can be Stationed, Moved, Rotated and/or Swept, in any desired sequence, individually or in any desired combination, to Interrupt S/PS/SN nerves to Treat a Medical Condition of a Patient.

However, if a thermal energy delivery device is used in an air or biocompatible gas environment, without the infusion or a spray of a sterile, biocompatible irrigation fluid, it should be used at the relatively low energy levels described below to avoid excessive thermal damage to the Target Nerve Tissue, or adjoining tissues.

If used in an air or biocompatible gas environment, we found that the cannula/optical fiber device described above can be improved by providing a space between the exterior of the optical fiber and the inner surface of the cannula for infusion of a sterile, biocompatible fluid, such as saline, water, a cooled gas, preferably a cryogenically cooled gas, such as CO2 or nitrogen, to cool the optical fiber and the Target Nerve Tissue.

A luer fitting, in fluid communication with the space between the exterior of the optical fiber and the interior of the cannula, may be attached to the handpiece or the cannula, as described above, can be used to infuse the cold fluid. Cooling the optical fiber and the Target Nerve Tissue enables a substantially greater level of laser energy or other Source of Thermal Energy to be safely used to Interrupt S/PS/SN nerves of the Target Nerve Tissue.

Other variations of the above described devices can be made and other Sources of Thermal Energy to Treat a variety of Medical Conditions of Patients can be used, without departing from the principles set forth herein and without limiting the intent and scope of the present invention.

### BRIEF DESCRIPTION OF THE DRAWINGS

FIG. 1 is an external, side view of the first embodiment of the device of the present invention, with an expanded, partial, cross-sectional, side view of the distal end portion of the device.

FIG. 2 is a partial, cross-sectional, side view of the distal end portion of the second embodiment of the device of the present invention.

FIG. 3 is a partial, cross-sectional, side view of the distal end portion of the third embodiment of the device of the present invention.

FIG. 4 is a partial, cross-sectional, side view of the distal end portion of a variant of the device of FIG. 2.

FIG. 5 illustrates the laser energy emission area resulting from Stationing and Rotating the embodiment of the device of FIG. 2.

FIG. 6 is a cross-sectional, side view of the distal end portion of a further variant of the device of FIG. 2.

FIG. 7 is a partial, cross-sectional, side view of another variant of the device of FIG. 6.

FIG. 8 is a partial, cross-sectional side view of another variant of the device of FIG. 2.

FIG. 9 is a cross-sectional, end view at plane A-A of the device of FIG. 8.

FIG. 10 is a partial, cross-sectional, side view of yet another variant of the device of FIG. 7.

FIG. 11 is a cross-sectional, end view at plane B-B of the device of FIG. 10.

FIG. 12 is a partial, cross-sectional, side view of further variant of the device of FIG. 10.

FIG. 13 is a cross-sectional, end view at plane C-C of the device of FIG. 12.

FIG. 14 is a partial, cross-sectional, side view of the handpiece and luer fittings of the devices shown in FIGS. 8, 10 and 12.

FIG. 15 is a cross-sectional, end view of the collar and three luer fittings of an embodiment of the device of the FIG. 12.

FIG. 16 is a schematic representation of a method of use of the device of the present invention.

FIG. 17 is a schematic representative of another method of use of the device of the present invention.

FIG. 18 is a schematic representation of another method of use of the device of the present invention.

FIG. 19 is a schematic representation of the combined methods of use of the devices of FIGS. 17 and 18.

FIG. 20 is a partial, external, side view of four other optical fiber/cannula devices.

FIG. 21 is a partial, external, side view of four improved optical fiber/cannula embodiments of the device of the present invention.

FIG. 22 is a cut-through, oblique, end view of a main renal artery of the kidney.

FIG. 23 is a cut-through, end view of a branch of the bronchi of the lung.

FIG. 24 is an external, posteroinferior view of the heart.

FIG. 25 is an external, side view of the stomach, duodenum, colon, liver and pancreas.

FIG. 26 is an external, inferior view of the brain.

FIG. 27 is an external, side view of a vertebra.

FIG. 28 is a cut-through top view of a vertebra and spinal disc.

FIG. 29 is an external, top view of the sacrum of the spine.

FIG. 30 is a partially cut-through, side view of the testes.

### DETAILED DESCRIPTION OF THE INVENTION

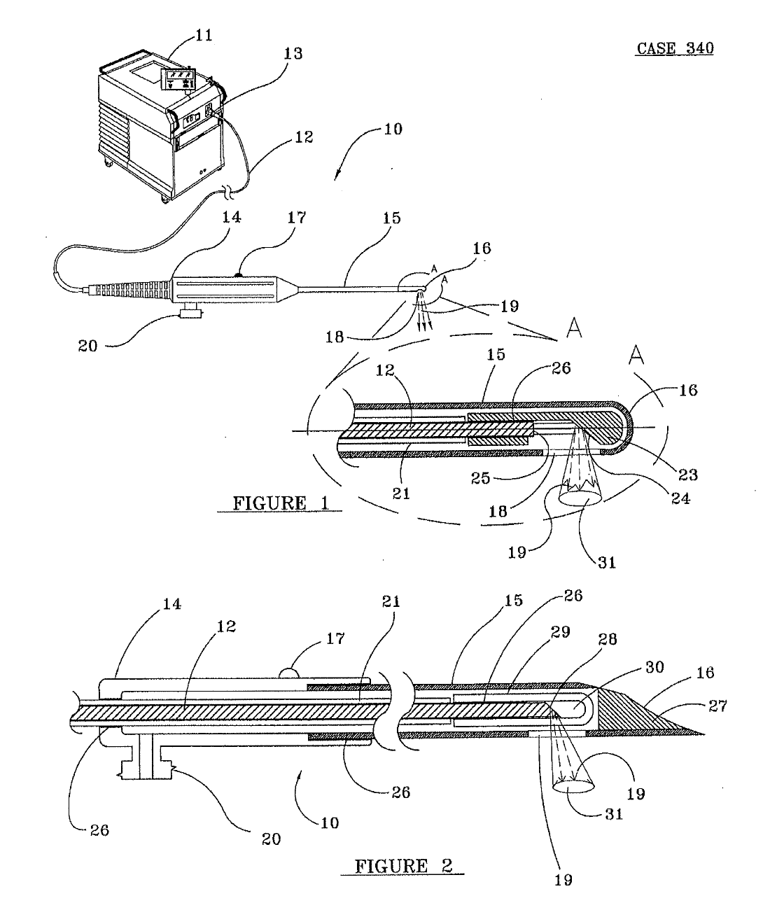

The first embodiment of side firing device 10 of the present invention is illustrated in FIG. 1. In this embodiment, device 10 is comprised of laser energy source 11 and optical fiber 12. Connector 13 optically couples optical fiber 12 to laser energy source 11. Optical fiber 12 is fixedly and sealingly attached within the proximal end of handpiece 14 by adhesive 26, as known in the art, and extends through a hollow, longitudinal, passageway (not separately shown) in handpiece 14 and is in fluid communication with hollow metal or plastic cannula 15, preferably of medical grade stainless steel, whose proximal end is fixedly attached by adhesive 26 within the distal end of handpiece 14. Cannula 15 can be rigid, semi-rigid, flexible, or pliant.

The distal end 16 of cannula 15, as shown in FIG. 1, is rounded. Distal end 16 of cannula 15 may also be blunt, sharp, double-bevel needle-shaped, trocar shaped or of any other desired shape, as known in the art. Using a needle-like or sharp-ended cannula within a patient entails considerable risk to the patient, should be used under endoscopic, ultrasound or x-ray imaging and requires greater care by the surgeon.

Alternatively, optical fiber 12 may be removeably and sealingly attached within the proximal end of handpiece 14 by a compression fitting (not separately shown), as known in the art, enabling side firing device 10 to be removed, cleaned, sterilized and reused, if desired.

Button 17 on handpiece 14, in this embodiment, is preferably positioned on the side of handpiece 14 opposite the side from which the emission of laser energy occurs through laser energy emission port 18 in cannula 15, as shown by arrows 19, resulting in laser energy spot area 31 on or within a Target Nerve Tissue. While button 17 may also be positioned on the side of handpiece 14 from which the emission of laser energy occurs, button 17 will be less able to be visualized during use.

Luer or other fluid connector fitting 20, which is fixedly attached within and extends through the wall of handpiece 14, is in fluid communication with the longitudinal passageway (not separately shown) in handpiece 14, hollow cannula 15 and port 18 positioned over the source of emission of laser energy. Luer fitting 20 enables a sterile, biocompatible fluid, such as saline or water, to be infused through longitudinal passageway (not separately shown) in handpiece 14 into hollow cannula 15, to clean and cool the laser energy emitting surface of side firing device 10 and cool the Target Nerve Tissue.

As shown in the cut-through, expanded view of the distal end portion of device 10, buffer coating 21 and any optional polymer cladding (not separately shown) of optical fiber 12 have been removed from the distal end portion of optical fiber 12, which extends into cavity 22 in hollow metal tip 27. Metal tip 23 is fixedly attached to the bared distal end portion of optical fiber 12 by adhesive 26, crimping (not separately shown) or both, or by other means known in the art.

As illustrated, cavity 22 in metal tip 23 is formed with a reflective, inclined surface 24 opposite distal end face 25 of optical fiber 12. Reflective surface 24 of metal tip 23 is inclined at an angle of about 35° to 50°, preferably about 45°, to reflect the laser energy from inclined reflective surface 24 at an angle of about 90° from the optical axis of optical fiber 12, according to Snell's law, out of port 18, as shown by arrows 19.

Metal tip 23 can be made entirely of a metal highly reflective to the wavelength of laser energy to be used, such as highly pure gold or silver, or metal tip 23 can be made of a material such as medical grade stainless steel, which is plated with a highly reflective metal, such as highly pure gold or silver with a thickness of about 5 thousandths of an inch or more, or coated with a dielectric highly reflective to the wavelength of laser energy to be used, as known in the art.

Alternatively, an insert (not separately shown) with a thickness of about 10 to 20 thousandths of an inch or more of a metal highly reflective to the wavelength of laser energy being used, such as highly pure gold or silver, may be force-fitted or attached by an adhesive, or both, in a recess (not separately shown) in the distal end of the cavity 22 in metal tip 23.

Polished copper, brass, aluminum or stainless steel, which cost less than gold or silver, may also be used. However, stainless steel is not a highly efficient reflector, and copper and aluminum are not as reflective as gold or silver and are subject to tarnish and/or oxidation, which reduces their reflectivity.

95.5% pure Silver is about 97% reflective at wavelengths of about 500 to 2400 nm, and about 95.5% reflective at 430 nm. 95.5% pure Gold is less than 50% reflective below wavelengths of 500 nm, 81.7% reflective at 550 nm, 91.9% reflective at 600 nm, 95.5% reflective at 650 nm and about 97% reflective at 700 nm and longer wavelengths. Highly pure platinum is extremely expensive and is only 71.4% to 81.8% reflective at wavelengths of 500 to 2000 nm and is 88.8% reflective at 3000 nm. Highly pure silver is preferred, because it is highly reflective and is considerably less expensive than gold or platinum.

However, for greater durability, a lower cost of manufacture and resistance to erosion by the emission of laser energy, metal tip 23 is preferably made entirely of very pure gold or silver, preferably of very pure silver with a purity of about 95.5%. For comparison, “Sterling” silver is 92.5% pure.

The second embodiment of side firing device 10 of the present invention is shown in FIG. 2. In this embodiment, distal end 16 of hollow cannula 15 is shaped like the distal end of a double beveled syringe needle, which cuts rather than puncturing or making a hole through the skin, hastening healing and reducing bleeding and the risk of an infection. To prevent tissue from lodging in the opening at distal end 16 of cannula 15, plug 27 of adhesive 26 or other material, preferably heat resistant to any stray laser energy, may be used to fill distal end 16 of cannula 15.

Distal end 16 of hollow cannula 15 can also be blunt, round, conical or any other desired shape, as the use of a sharp or needle-like device within a patient requires imaging during its use and great care by the surgeon.

Buffer coating 21 and any optional polymer cladding have been removed from the distal end portion of optical fiber 12, and the distal end of optical fiber 12 has been ground and polished into beveled, distal end surface 28 at an angle of about 35° to 45° with respect to the longitudinal axis of the optical fiber. The beveled, distal end portion of optical fiber 12 is sealingly encased within hollow, closed-ended capillary tube 29, which creates air pocket 30 opposite beveled, distal end surface 28 of optical fiber 12. Air pocket 30 has a lower refractive index than that of the core of optical fiber 12, which is necessary for total internal reflection or “TIR” of laser energy at double the bevel angle of distal, beveled end surface 28, according to Snell's Law.

According to common wisdom in the medical laser field, the most effective bevel angle of an optical fiber for total internal reflection of laser energy is 37°. Contrary to common wisdom, distal end surface 28 of optical fiber 12 is preferably beveled at an angle of about 40° to 41°, which we have discovered by testing various bevel angles at 1° intervals, to be the most efficient bevel angle of an optical fiber for total internal reflection of laser energy at relatively high power levels.

If beveled, distal end surface 28 of optical fiber 12 is ground and polished at an angle less than 40°, the laser energy will be less optimally reflected and more scattering of laser energy will occur. If distal end surface 28 of optical fiber 12 is beveled at an angle greater than 42°, the transmission of laser energy will be substantially lower.

Capillary tube 29 typically has a wall thickness of 500 microns or more, as it may be eroded during use, causing device 10 to fail. The proximal end portion of closed-ended capillary tube 29 may be fixedly and sealingly attached to the bared distal end portion of optical fiber 12 by thermal fusion (not separately shown) or by adhesive 26, neither of which extend into the area of laser energy emission from beveled, distal end surface 28 of optical fiber 12. While not preferred, if capillary tube 29 is fused to optical fiber 12 near beveled, distal end surface 28 of optical fiber 12, care must be taken to avoid deforming beveled distal end surface 28 of optical fiber 12 by exposure to high glass fusing temperatures.

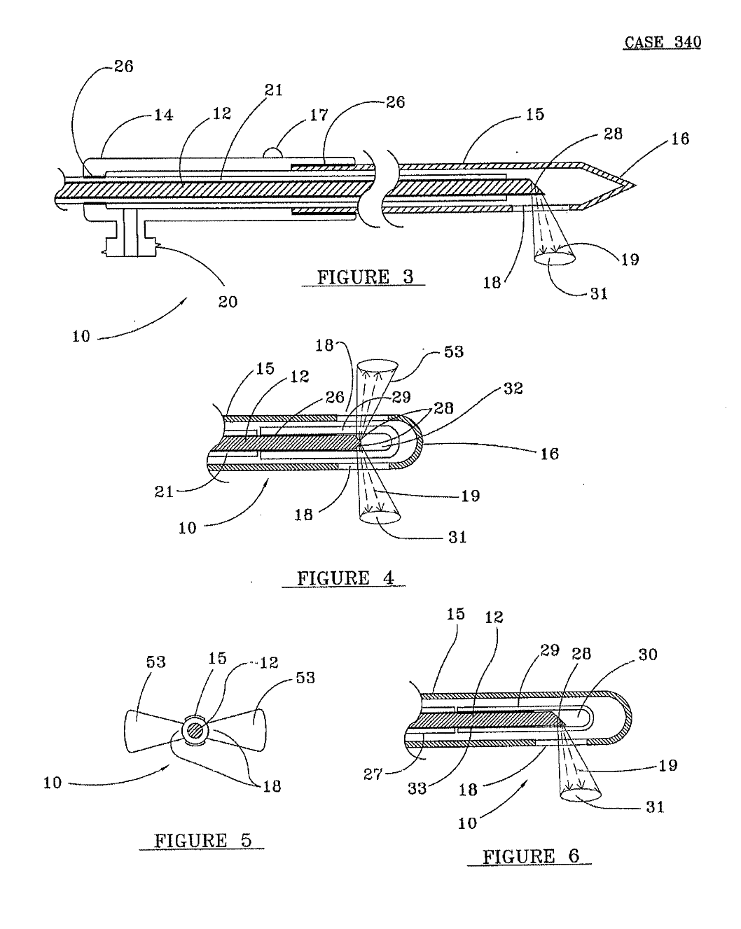

FIG. 3 illustrates the third embodiment of side firing device 10 of the present invention. In this embodiment, no capillary tube 29 is utilized to sealingly encase the beveled, distal end surface 28 of optical fiber 12. As a result, no air pocket is created opposite beveled, distal end surface 28 of optical fiber 12.

Laser energy at wavelengths of 1400 to 1500 nm and 1800 to 11,000 nm is highly absorbed by aqueous liquids, such as sterile saline or water, which are commonly used as an irrigation fluid in endoscopic procedures. If ten watts or more of laser power at these wavelengths is transmitted through optical fiber 12, such wavelengths of laser energy cause a steam and/or gas bubble to form, with each pulse of laser energy, opposite beveled, distal end surface 28 of optical fiber 12, from the vaporization of the aqueous irrigation liquid, blood, other body fluids and/or tissue.

The refractive index of the steam and/or gas bubble opposite beveled, distal end surface 28 of optical fiber 12 is sufficiently lower than the refractive index of the quartz or fused silica core of optical fiber 12, to enable the laser energy to be totally internally reflected from beveled, distal end surface 28 of optical fiber 12, laterally from the axis of optical fiber 12 at an angle of 80° to 82°, as shown by arrows 19, according to Snell's Law, and the balance of the pulse of laser energy passes through the stream and/or gas bubble to the Target Nerve Tissue. Consequently, no capillary tube 29 need be disposed over the 40° to 41° beveled, distal end surface 28 of optical fiber 12 to create an air interface and TIR.

However, laser energy at 300 to 1400 and 1500 to 1800 nm cannot be used through device 10 of this third embodiment of the present invention, as such wavelengths are not highly absorbed by water and no steam and/or gas bubble with a refractive index lower than the core of optical fiber will be formed, and the laser energy will be emitted straight-ahead.

As shown, distal end 16 of cannula 15 is pointed or conically shaped. As mentioned above, the use of a pointed or sharp-ended cannula in a patient entails significant risk and should be used under endoscopic, ultrasound or x-ray viewing.

FIG. 4 illustrates side firing device 10 in which the distal end of optical fiber 12 is beveled into a chisel like shape, with each distal, beveled end surface 28 at an angle of 40° to 41° from the axis of optical fiber 12. The proximal end of capillary tube 29 is fixedly attached to the bared distal end surface of optical fiber 12, buffer coating 21 and any polymer cladding (not separately shown) having earlier been removed from the distal end portion of optical fiber 12.

Capillary tube 29 creates air pocket 30 opposite both distal beveled end surfaces 28 of optical fiber 12, necessary for TIR of laser energy, according to Snell's Law. As indicated by arrows 19, laser energy emitted from both ports 18 in cannula 15 exits at an angle of about 80° to 82° from the axis of optical fiber 12, simultaneously creating laser energy spot areas 31.

In this embodiment, to achieve the same effect on a Target Nerve Tissue, the power level of emitted laser energy must be doubled.

As illustrated in FIG. 4, side firing device 10 emits laser energy simultaneously, for example, at 3 o'clock and 9 o'clock and, by doubling the level of laser energy, creates bowtie-shaped laser energy pattern 35 by simply Rotating device 10 back and forth through an arc of about 90°. This device is used within a rigid metal or plastic outer tube or cannula, the tube or cannula must have two ports for emission of laser energy, each positioned opposite one of beveled, distal end surfaces 28 of optical fiber 12.

FIG. 5 illustrates the laser energy emission pattern 35 from the side firing devices of FIGS. 1-3 (and those of the side firing devices of FIGS. 4, 6-8, 10 and 12 described below) if, for example, side firing device 10 is Stationed first to emit laser energy at 3 o'clock (button 17 being positioned at 9 o'clock) and, while laser energy, at a desired level for a desired period of time, depending on the volume and type of Target Nerve Tissue to be Interrupted, is emitted, side firing device 10 is repetitively Rotated through an arc of 90° to 120°. Then, side firing device 10 is, for example, Stationed to emit laser energy at 9 o'clock (with button 17 positioned at 3 o'clock) and, while laser energy as described above is emitted, side firing device 10 is repetitively Rotated through an arc of 90° to 120°, at the rate of Rotation described above, preferably at a rate of about one cycle per second. This creates a “bowtie-shaped” laser energy emission pattern.

The same “bowtie” shaped laser energy pattern occurs from the Stationing and Rotating of the device of FIG. 4, without the need to position it to emit first, for example, at 3 o'clock, and then at 9 o'clock, using twice the level of laser energy.

The above applications of bowtie-shaped laser energy emission pattern 35 to denervate S/PS/SN nerves avoids damage to tissues above and below the Target Nerve Tissue.

FIG. 6 illustrates a fifth improved embodiment of side firing device 10 of FIG. 2. In this embodiment, the end portion of bared optical fiber 12 is fixedly and sealingly encased within a distally closed-ended capillary tube 29, which has a substantially thinner wall thickness than the typical 500 micron or larger wall thickness of capillary tube 29 shown in FIG. 2. The wall thickness of capillary tube 29 in this embodiment is preferably about 350 microns or less, which reduces the amount of cylindrical lensing that occurs and converges the divergent output of laser energy from beveled, distal end surface 28 of optical fiber 12 at a closer point, providing an effectively wider angle of divergence at a given distance from laser energy emission port 18, as illustrated by arrows 19. This results in a significantly larger laser energy spot area 31 on or within a Target Nerve Tissue than laser energy spot area 31 shown from side firing device 10 of FIG. 2.

However, this embodiment of the present invention is preferably used at low power levels of laser energy, for example, to Interrupt S/PS/SN nerves of the renal arteries, Interrupt S/PS/SN nerves of the bronchi, and Interrupt S/PS/SN nerves of other Target Nerve Tissues.

Side firing device 10 of FIG. 6 should not be used to Treat a Medical Condition of a Patient which requires the emission of a very high level of laser energy power for a substantial period of time, such as 40 to 100 watts for 20 to 40 minutes or longer, as thinner capillary tube 29 is more likely to be degraded by hydrothermal erosion and laser energy back reflected from the Target Nerve Tissue, causing device 10 to fail. Hydrothermal erosion is created by the formation of a steam bubble, when each pulse of laser energy at wavelengths of 1400 to 1500 and 1800 to 11,000 nm is emitted, and a powerful acoustic shock wave is created by the collapse of the bubble, which can erode capillary tube 29.

In addition to creating larger spot area 31, another benefit of device 10 of FIG. 6 is that optical fiber 12, handpiece 14, cannula 15 and emission port 18 of device 10 may be rotated through an arc of only about 90°, instead of up to about 120°, to Interrupt S/PS/SN nerves of a Target Nerve Tissue.

FIG. 7 illustrates a sixth embodiment of side firing device 10 of the present invention. In order for side firing device 10 such as shown in FIG. 2 to be bent at an angle of up to 90° or more when used, for example, (a) through a conventional guiding catheter to access, for example, at an angle of about 90°, the main renal arteries of the kidneys, from the internal aorta, (b) through a flexible bronchoscope to access branches of the bronchi, (c) through a rigid endoscope whose distal end portion of about 5 to 10 cm is flexible and can be articulated or bent by wires or other means up to 90° or more, as known in the art, and (d) to access the top, back, bottom and front exterior surfaces of an artery, vein, bronchi, gland, organ, duct, bone or a joint, optical fibers with a core diameter of 500 microns or larger may not be sufficiently flexible to be used through such devices.

Common wisdom in the laser field is that only optical fibers with core diameters of 500 microns or larger can be effectively used to transmit up to 100 watts or more of laser power, and have a sufficient surface to be beveled to effectively reflect laser energy at an angle of 70° to 90°. Contrary to common wisdom, by testing optical fibers of successively smaller diameter, we discovered that optical fibers with a core diameter of no more than 350 microns could be effectively beveled at their distal end portion and used with appropriate cladding materials through bends of up to 90° or more with up to about a 95% laser energy transmission efficiency, provided the bend radius is not less than 1 to 1.5 cm, as will be explained later.

As a result, as seen in FIG. 7, we created the smallest side firing device 10 ever made, with an O.D. of only about 1.5 mm, compared to prior art side firing devices 10 with an O.D. of 2 to 2.5 mm, enabling these smaller diameter side firing device 10 to be used in arteries, veins, bronchi, ducts and surgically created passageways, which may be cannulated, with an I.D. of 1.5 mm or larger.

As seen, optical fiber 12 has a core diameter of 350 microns, whose distal end surface 28 has been beveled at an angle of 40 to 41° from the longitudinal axis of optical fiber 12. Buffer coating 21 and any optional polymer cladding (not separately shown) has been removed from the distal end portion of optical fiber 12. Capillary tube 29 fixedly and sealingly encases the distal end portion of optical fiber 12, as described above, creating air pocket 30 to enable total internal reflection of light to occur through port 18, as shown by arrows 19.

For use at relatively high laser energy power levels, as shown, capillary tube 29 can have a wall thickness of about 500 microns. For use at relatively lower levels of laser energy power, capillary tube 29 can have a wall thickness of 350 microns or less, as shown in FIG. 4.

The proximal end portion of capillary tube 31 can be fixedly attached to bared optical fiber by thermal fusion (not separately shown), by adhesive 26 or both. Adhesive 26 is preferably made of a material with a high melting point, which meets USP Class VI specifications for use in medical devices and which is substantially transparent to the wavelengths of laser energy commonly used in medical procedures, such as KTP, diode, Nd:YAG, Thulium:YAG and CTH:YAG or Holmium lasers, so as not to absorb laser energy and melt, allowing capillary tube 29 to move with respect to optical fiber 12 and be dislodged therefrom.

An adhesive 26 which meets all of the above requirements is an optically transparent, low viscosity epoxy adhesive. Such adhesives are commercially available. Adhesive 26 preferably has a relatively high melting point, is optically transparent to and does not absorb the wavelengths of laser energy commonly used in medical procedures, 980 mm diode, 1064 nm Nd:YAG, 1470 nm diode, or 2100 nm CTH:YAG laser energy, not absorbing more than an average of 6% of such laser energy. Illustrative of such adhesives are the U.S.P. Class VI approved, two component epoxide epoxy resins and the like.

As a safety measure, heat shrinkable tubing 32 is shrunk over the distal end portion of buffer coating 21 and the proximal end portion of capillary tube 29, terminating before laser energy emission port 18. Adhesive 26 can also be optionally used to fixedly attach heat shrinkable tubing 32 in place, as an additional safety measure, to help prevent the accidental separation of capillary tube 29 from optical fiber 12.

FIG. 8 illustrates the seventh embodiment of device 10 of the present invention. In this embodiment, flexible plastic, round, hollow, doubled-walled, multi-channel tube 33 extends from about the distal end of or within the distal end of handpiece 14 (not separately shown) over optical fiber 12 and, as shown, terminates just before the proximal end of heat shrunk tubing 32.

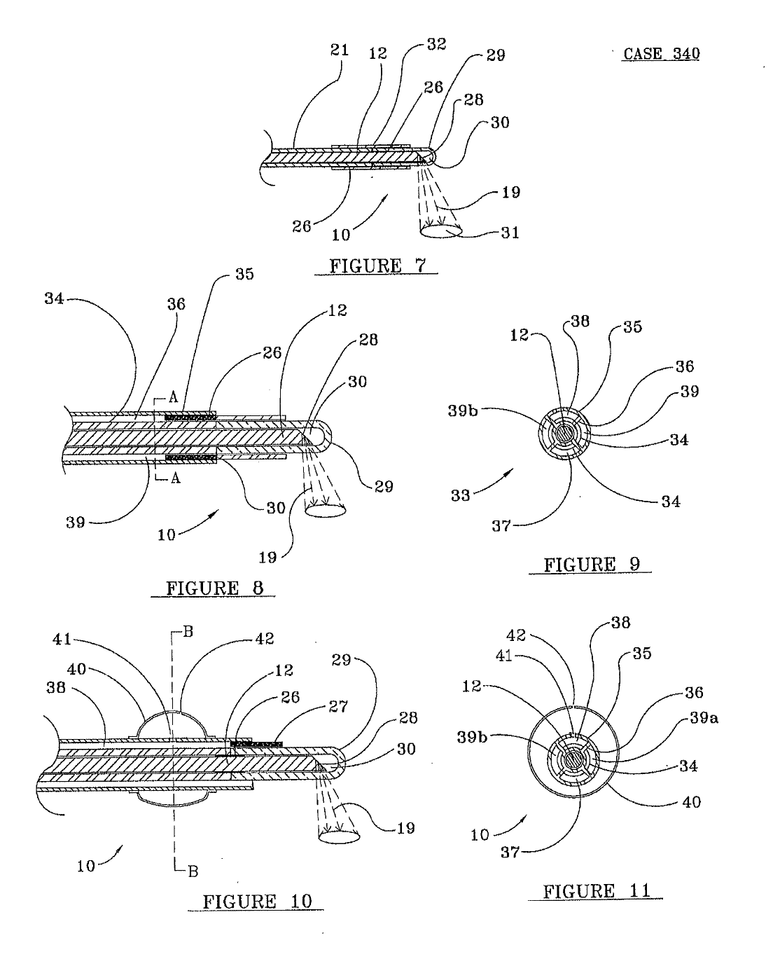

Round, hollow, double-walled, multi-channel tube 33 consists of round inner wall 34 and round outer wall 35. The I.D. of inner wall 34 of tube 33 is just slightly larger than the O.D. of optical fiber 12. To space inner wall 34 apart from outer wall 35, tube 33 is extruded with two or more longitudinally extending ribs 36 (not separately shown in FIG. 8). Preferably 4 ribs 36 are extruded, creating 4 channels 37, 38, 39a and 39b (not separately shown), as described below in FIGS. 9, 11 and 13.

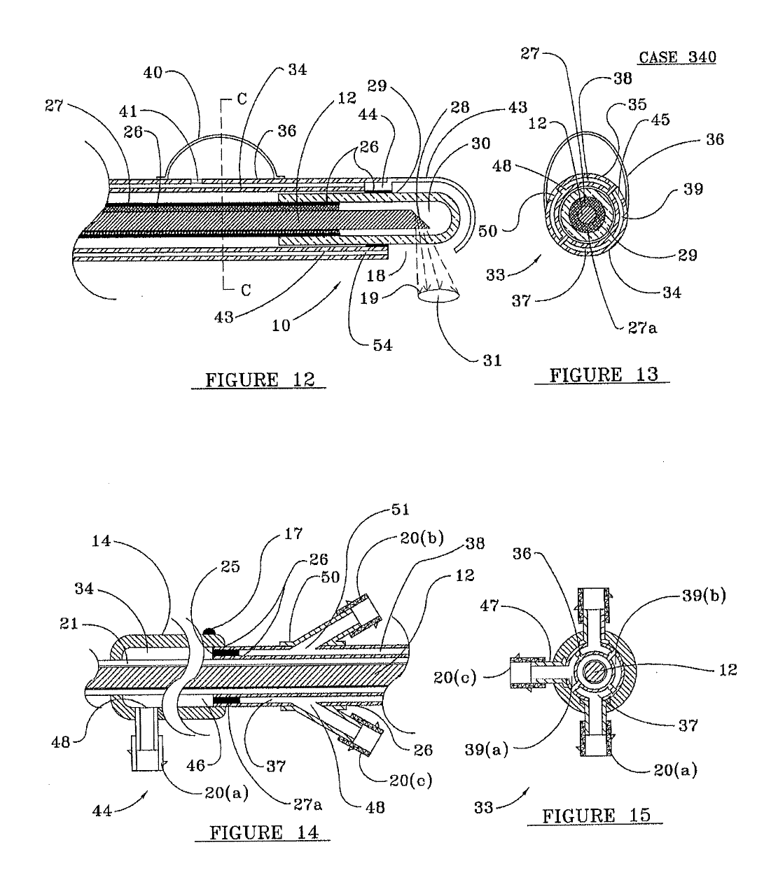

FIG. 9 illustrates the construction of flexible, round, hollow, double-walled, multi-channel tube 33 at a plane A-A of FIG. 8. Inner wall 34 of tube 33 is circular with an I.D. just slightly larger than that of optical fiber 12 of the devices of FIGS. 1-4 (and the smaller diameter optical fibers 12 of FIG. 7 described above and those of FIGS. 10 and 12 described below).

In this embodiment, for example, four ribs 36 extend longitudinally through and separate inner wall 34 from outer wall 35 of tube 33, with ribs 36 preferably located at 2, 4, 8 and 10 o'clock, creating channels 37, 38, 39(a) and 39(b), as described below in FIGS. 10 and 11.

Channel 37 may be in fluid communication with fluid passageway in handpiece 14 and luer fitting 20 (neither of which are separately shown), and a sterile biocompatible fluid, such as saline or water, can be infused through channel 37 to clean and cool the laser energy emitting surface of capillary tube 29 and the Target Nerve Tissue.

FIG. 10 illustrates the eighth embodiment of device 10 of the present invention. In this embodiment, double walled, hollow tube 33 extends from about the distal end or within the distal end of handpiece 14 (not separately shown), over optical fiber 12 and heat shrunk tubing 32 and co-terminates with the distal end of heat shrunk tubing 32, proximal to the area of laser energy emission from capillary tube 29, as shown by arrows 19.

Balloon 40 eccentrically encases a portion of the proximal end portion of hollow, double-walled tube 33. The wider portion of eccentric balloon 40 presses the laser energy emitting surface of capillary tube 29 closer to the Target Nerve Tissue and minimizes the loss of laser energy in vaporizing any intervening aqueous irrigation fluid. Irrigation fluid infused through channel 37 also forces blood and other bodily liquids (not separately shown) away from the laser energy emission area of capillary tube 29, as shown by arrows 19.

A sterile, biocompatible irrigation fluid, such as saline or water, may also be infused through channel 38 and exits vent 41 in outer wall 35 to inflate balloon 40. In this embodiment, balloon eccentric 40 has one or more apertures or holes 42. When the irrigation fluid is infused through channel 38 to inflate balloon 40, one or more holes 42 enable air to be purged from channel 38 and escape from balloon 40. When irrigation fluid is seen exiting hole or holes 42, the operator knows the air has been purged from channel 38 and balloon 40 and device 10 may be safely inserted into a patient, to avoid an air embolism.

In this embodiment, channels 39(a) and 39(b) are not used, and the proximal ends of channels 39(a) and 39(b) and the distal ends of channels 38, 39(a) and 39(b) are closed by plugs 27 of adhesive 26 or other material known in the art.

Balloon 40 can also be concentric to center device 10 in a blood vessel, duct, hollow organ or surgically created passageway and insure an equal amount of laser energy will be emitted to the inner surface of the blood vessel, duct, hollow organ or passageway at each area of laser energy emission, where this effect is desired. Balloon 40 can also be back-mounted to force the energy emission port 19 of device 10 close or closer to the Target Nerve Tissue.

FIG. 11 illustrates the construction of double-walled, hollow tube 33 at plane B-B of FIG. 10. In this embodiment, outer wall 35 of double-walled, hollow tube 33 has vent 41, allowing a sterile, biocompatible fluid, such as saline or water, to be infused through channel 38 and exit through vent 41 to inflate eccentric (or concentric or back-mounted) balloon 40 which encases the portion of device 10 proximal to its laser energy emitting surface. Balloon 40 has one or more tiny holes 42 to enable air to be purged from channel 38 and allow excess sterile, biocompatible fluid to escape balloon 40, indicating by its appearance from hole or holes 42, that all of the air in channel 38 has been purged.

FIG. 12 illustrates the ninth embodiment of device 10 of the present invention. In this embodiment, inner wall 34 of double-walled, hollow tube 33 is circular to accept capillary tube 29 sealingly encasing optical fiber 12, which are disposed eccentrically within double-walled, hollow tube 33, by the thicker walled plug 27 versus that of thinner walled plug 27(a), positioning the laser energy emitting surface of capillary tube 29 closer to the Target Nerve Tissue. Vent 41 in outer wall 36 allows a sterile, biocompatible fluid, for example, saline or water, to be infused through channel 38 and vent 41 to inflate balloon 40.

Balloon 40 is mounted on the back side of hollow, double-walled tube 33, opposite the side of device 10 from which laser energy is emitted from capillary tube 29, as shown by arrows 19. The inflation of balloon 40 forces side firing device 10 close to the Target Nerve Tissue, and the infusion of fluid through channel 37 forces blood away from the path of laser energy emission.

FIG. 13 illustrates the construction of device 10 at plane C-C of FIG. 12. In this embodiment, fluid infused through channel 38 and vent 41 in outer wall 35 to inflate balloon 40, exits balloon 40 through vent 45 in outer wall 35 into return channel 39(a), through luer filling 20 (not separately shown) and flows to a drain or a collection bottle (not separately shown).

Alternatively fluid return channel 39(a) can empty into a plastic tube which can be clamped shut, as known in the art, when balloon 40 has been inflated, and which can be unclamped and a vacuum applied to empty balloon 40 and channels 38 and 39(a) when the procedure has been completed to enable device 10 to be safely removed from the patient. The distal ends of channels 38, 39(a) and 39(b) remain closed by plugs 27.

Optional end cap 43, which may be made of metal or a rigid plastic, as shown, is rounded to provide an atraumatic distal end of device 10. End cap 43 may be blunt, sharp, pointed or of any other desired shape. Circular flange 44 of end cap 43 is fixedly attached between outer wall 35 and inner wall 34 of hollow, double-walled tube 33 by adhesive 26 and effectively plugs the distal ends of channels 38, 39(a) and 39(b).

FIG. 14 illustrates how luer fillings 20 (a) and (b) enable a sterile, biocompatible fluid to pass through luer fitting 20(a) into passageway 46 in handpiece 14 and flow through channel 37 of double-walled, hollow tube 33 to clean debris from capillary tube 29 and cool the laser energy emitting surface of capillary tube 29 and the Target Nerve Tissue. Such fluid also passes through luer fitting 20(b) and flows through channel 38 of hollow, double-walled tube 33 and vent 41 in outer wall 35 to inflate balloon 40. Excess fluid used to inflate balloon 40 exits balloon 40 through vent 45 (not separately shown) in outer wall 35 and flows through channel 39(a) and luer fitting 20(c), as described above, and as seen earlier in FIG. 15).

As shown, luer fitting 20(a) is fixedly attached to fluid line 47 by adhesive 26 or other adhesive known in the art. Fluid line 47 is fixedly attached to opening 48 in the body of handpiece 14 by adhesive 26 or other adhesive known in the art. Luer fitting 20(b) is fixedly attached by adhesive 26 or other adhesive known in the art to fluid line 49, whose distal end is cut at an angle or bias 49, as shown.

Fluid line 47 is extruded with circular flange 50, which is fixedly attached by adhesive 26 or other adhesive known in the art to outer wall 35 of hollow, double-walled tube 33, over opening 51 in outer wall 35, which is in fluid communication with channel 38. Plugs 27 close the proximal ends of channels 37, 38 and 39, which may consist of adhesive 26 or other adhesive known in the art. Luer 20(c) is not shown (See FIG. 15).

FIG. 15 illustrates an alternate construction of luer fittings 20(a)-(c) of side firing device 10. In this embodiment, double-walled, hollow tube 33 has four channels, 37, 38, 39(a) and 39(b), created by four ribs 36 between extending longitudinally through and separating inner wall 34 from outer wall 35, with luer fittings 20(a) and (b) each in fluid communication with channels 37 and 38 respectively. Flanges 50 of fluid lines 47 of luer fittings 20(a) and (b) are fixedly attached to outer wall 35 of double-walled, hollow tube 33 by adhesive 26 or other adhesive known in the art, in the manner described above with respect to luer fitting 20(b) of FIG. 14. Since luer fittings 20(a) and (b) are attached to double walled, hollow tube 33, instead of being attached to handpiece 14, luer fittings 20(a) and (b) do not interfere with the surgeon's handling of handpiece 14 of side firing device 10.

For ease of use, luer fittings (a)-(b) are disposed on outer wall 35 of double-walled, multichannel tube 33 a desired distance proximally from the proximal end of handpiece 14. Double-walled, multichannel tube 33 is in fluid communication through passageway 46 in handpiece 14 and is either fixedly attached within the distal end of connector 13 of FIG. 1 (not separately shown), or terminates at a point proximal from the point at which luer fittings 20(a) and (b) are located. The proximal ends of channels 37, 38, 39(a) and 39(b) are closed by plugs 27 of adhesive 26 or other adhesive known in the art (not separately shown). Likewise, the distal ends of channels 38, 39(a) and 39(b) are closed by plugs 27.

Any other number of ribs 36 may be used, creating any desired number of fluid channels, and ribs 36 may be positioned at any points, as desired, so long as none are in the path of laser energy emission from capillary tube 29 as shown by arrows 19.

As shown, inner wall 34 of hollow, double-walled tube 33 is circular and is positioned eccentrically with respect to outer wall 35 by plug 27(a) being thinner walled than thicker walled plug 27, disposing capillary tube 29 of side firing device 10 closer to the Target Nerve Tissue.

For support and greater strength, luer fittings 20(a)-(c) and fluid lines 47 may be fixedly attached by adhesive 26, or other adhesive known in the art, within rigid plastic or metal collar 52. In this embodiment, luer fittings 20(a)-(c) are disposed radially about double-walled, hollow tube 33 at 12, 6 and 9 o'clock. Alternatively, each of luer fittings 20(a)-(c) may be mounted separately at different points on outer wall 36 of tube 33, within or without individual collars 52 (not separately shown).

As mentioned earlier, 350 micron or smaller, thinner walled capillary tube 29 shown in FIG. 6 can be utilized in any of the embodiments of the present invention shown in FIG. 2 or 3, if side firing device 10 is to be used to emit a low level of laser energy, for example, about 10 watts, more or less. Likewise, capillary tubes 29 with a wall thickness greater than 350 microns, for example, about 450 to 600 microns, can be used in side firing devices 10 of FIGS. 7, 8, 10 and 12 if laser energy at higher levels is to be used, for example, at about 20 to 100 watts.

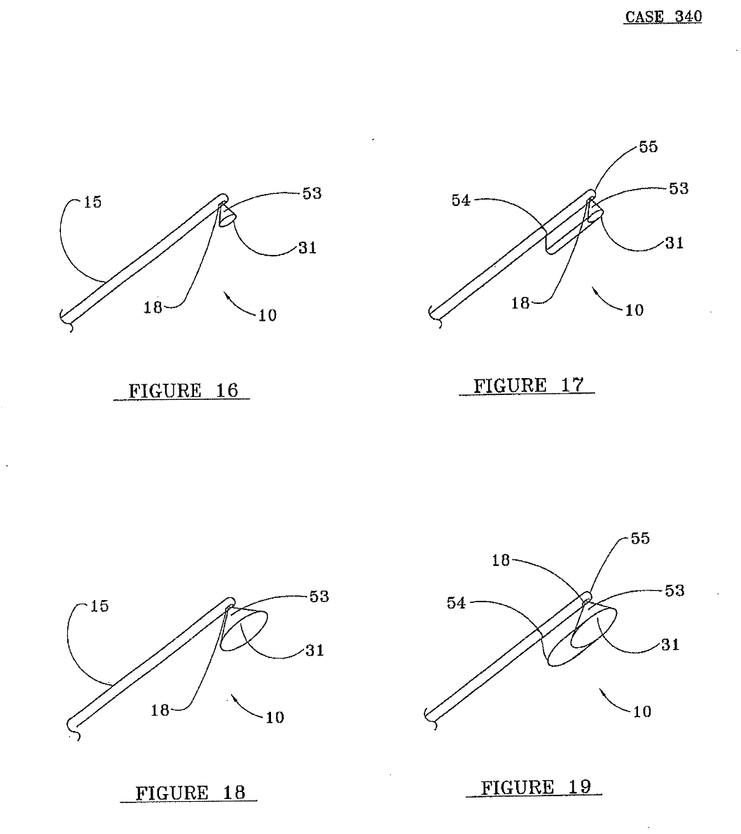

FIG. 16 illustrates laser energy emission pattern 53 and laser energy spot area 31, resulting from Stationing laser energy emission port 18 of side firing device 10, without moving device 10 or port 18, while laser energy is emitted at a desired energy level for a desired period of time, in a desired direction.

FIG. 17 illustrates larger laser energy emission pattern 53 and larger laser energy spot area 31 resulting from Stationing device 10 and Moving, by repetitively advancing and withdrawing side firing device 10 and laser energy emission port 18 at a desired rate of movement, from first point 54 to second point 55, while laser energy at a desired level for a desired period of time is emitted in a desired direction. The rate of Movement, the level of laser energy emitted and the time period of such emission is dependent, in the physician's discretion, upon the volume and depth of the Target Nerve Tissue to be Treated or the Interruption or Altering effect desired to be achieved on the Target Nerve Tissue.

FIG. 18, illustrates the laser energy emission pattern 53 and laser energy spot area 31, resulting from Stationing side firing device 10 and repetitively Rotating device 10 and laser energy emission port 18 through an arc, for example, of about 90 to 120°, while laser energy is emitted at a desired level and for a desired period of time, in a desired direction, at a rotation rate of about 0.5 to 2 seconds per cycle, preferably about one cycle per second, enabling the surgeon to mentally count, one thousand, two thousand, etc. per arc during the laser energy emission period.

FIG. 19 illustrates the larger laser energy emission pattern 53 and larger laser energy spot area 31 obtained by combining the above described Moving and Rotating processes of FIGS. 17 and 18, together or in any desired order or sequence, and Sweeping the Source of Thermal Energy, at a desired level of laser energy for a desired period of time, while laser energy is emitted in a desired direction, at a desired rate of Movement and Rotation from first point 54 to second point 55, to alter a large area or swath of Target Nerve Tissue.

As seen in FIGS. 16 and 17, laser energy diverges as it exits port 18, and the laser beam is narrow close to the laser energy's exit point from port 18. The benefit of combining the Moving process of FIG. 17 with the Rotation process of FIG. 18 in the Sweeping process described above as a wide area or swath of Target Nerve Tissue is irradiated, resulting in a more uniform Interruption of S/PS/SN nerves of a Target Nerve Tissue to Treat a Medical Condition of a Patient.

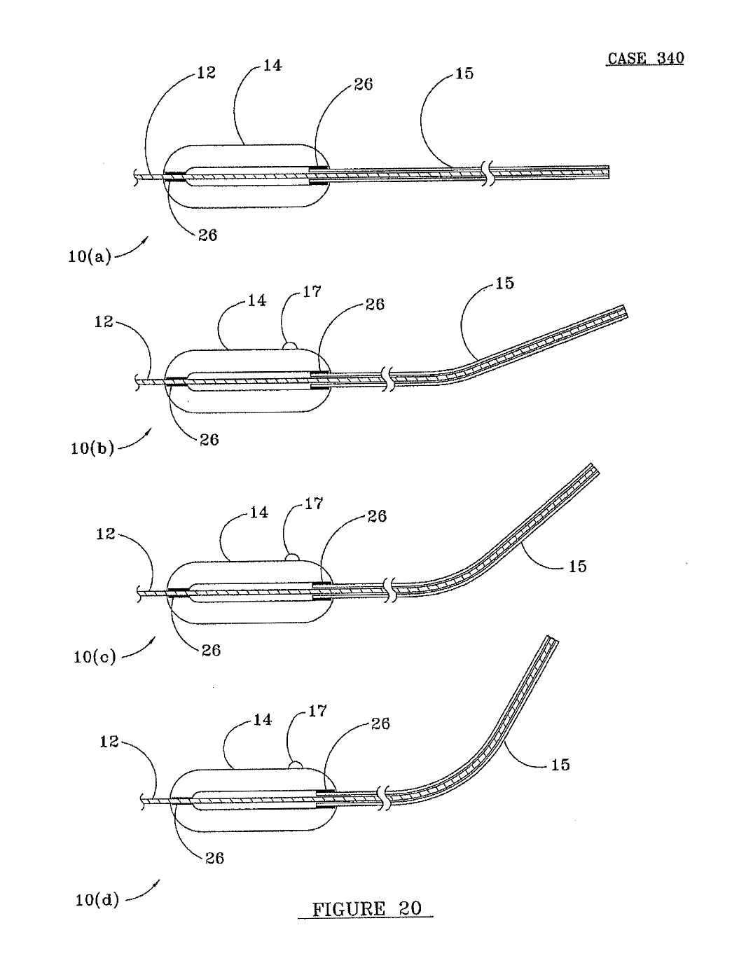

FIG. 20 illustrates four prior art laser energy delivery devices 10(a)-(d). The four devices 10(a)-(d) each contain optical fiber 12, which extends from a source of laser energy (not separately shown), passes through handpiece 14, is fixedly attached within the proximal or distal end of handpiece 14, closely fits within (or is fixedly attached by adhesive 26 to) the interior of rigid plastic or metal cannula 15, which is preferably made of medical grade, stainless steel or a nickel titanium alloy (nitinol).

Optical fiber 12 co-terminates at about the distal end of cannula 15, whose proximal end is fixedly attached within the distal end of handpiece 14. In each of devices 10(a)-(d), laser energy is emitted from the flat, distal end of optical fiber 12 straight ahead at an angle of 0° from the axis of the optical fiber.

Alternatively, optical fiber 12 can be removably attached to the proximal end of handpiece 14 by a compression nut as known in the art, enabling optical fiber 12 to be extended distally from the distal end of cannula 15 for cleaning and, if needed, clipping and cleaving to remove any deformed portion of optical fiber 12.

As can be seen, cannula 15 of device 10(a) is straight, to emit laser energy straight ahead at an angle of 0° from the axis of cannula 15. Cannula 15 of device 10(b) has a bend proximal to its distal end, as shown, at an angle of 20° from the axis of the main body of cannula 15. Cannula 15 of device 10(c) has a bend proximal to its distal end, as shown, at an angle of 40° from the axis of the main body of cannula 15, and cannula 15 of device 10(d) has a bend proximal to its distal end at an angle of 60° from the axis of the main body of cannula 15. Cannula 15 may also have a bend proximal to its distal end of 10°, 30°, 50° or any other desired angle from the axis of the main body of cannula 15.

Depending on the core diameter of optical fiber 12, the level of laser energy to be transmitted through optical fiber 12 and the temperature at which the cavity or lasing element of the laser is maintained, the radius of the bend must not be less than a certain radius, or leakage of laser energy through the quartz or fused silica cladding (not separately shown), which surrounds optical fiber 12, may occur. The cladding may contain a dopant, such as fluorine to lower its refractive index.

Escaping laser energy may cause cannula 15 to overheat and cause damage to cannula 15 and the instrument channel and optics of an endoscope (not separately shown), through which cannula 15 may be used. For example, if the cavity or lasing element of the source of laser energy 11 is cooled by a heat exchange device to a temperature of about 2 to 5° C., if optical fiber 12 has a core diameter of 365 microns and Holmium laser energy at a power level of 100 watts is to be transmitted through optical fiber 12, the bend radius preferably is not less than 1 cm.

If the cavity or lasing element (not separately shown) of the source of laser energy 11 is cooled by a chiller to a temperature close to freezing, about 0° C., if optical fiber 12 has a core diameter of 365 microns and Holmium laser energy at a power level of 10 watts is to be transmitted through optical fiber 12, the bend radius must not be less than 1.5 cm. As a result, bends in the distal end portion of cannula 15 must be made at a shallow angle.

While there is no button 17 on handpiece 14 of the 0° emitting or straight cannula 15, cannulas 15 bent at angles of 20°, 40°, 60°, as shown, or at any other desired angles, have button 17 on the side of handpiece 14 opposite from the direction of the bend, so the surgeon knows in what direction cannula 15 is being extended and the direction of laser energy emission. Button 17 should be raised and have a color different from that of handpiece 14, so it can be seen and be recognized by tactile feel by the surgeon.

Devices 10(a)-(d) of this FIG. 20, preferably with relatively small diameter optical fibers may be used where it is impractical to deliver laser energy from any of the side firing devices 10 described in FIG. 1-4, 6-8, 10 or 12.

A disadvantage of the devices shown in FIG. 20 is, in an air or gas Environment, such as CO2 gas or nitrogen, that these devices have no provision for delivering a sterile, biocompatible fluid to cool and clean the distal end of optical fiber 12 and the Target Nerve Tissue, as the devices 10(a)-(d) of FIG. 20 are typically used in an aqueous irrigation fluid, such as sterile water or saline. As a result, if side firing devices 10 of FIGS. 1-4, 6-8, 10, 12 or the devices of FIG. 20 are used in air or in a CO2 or nitrogen Environment, for example, in a laparoscopic or endoscopic procedure, a much lower level of laser energy, 0.05 to 3 watts, preferably 0.1 to 1.5 watts, should be used to prevent excessive heating and charring of the Target Nerve Tissue and thermal damage to adjacent tissues.

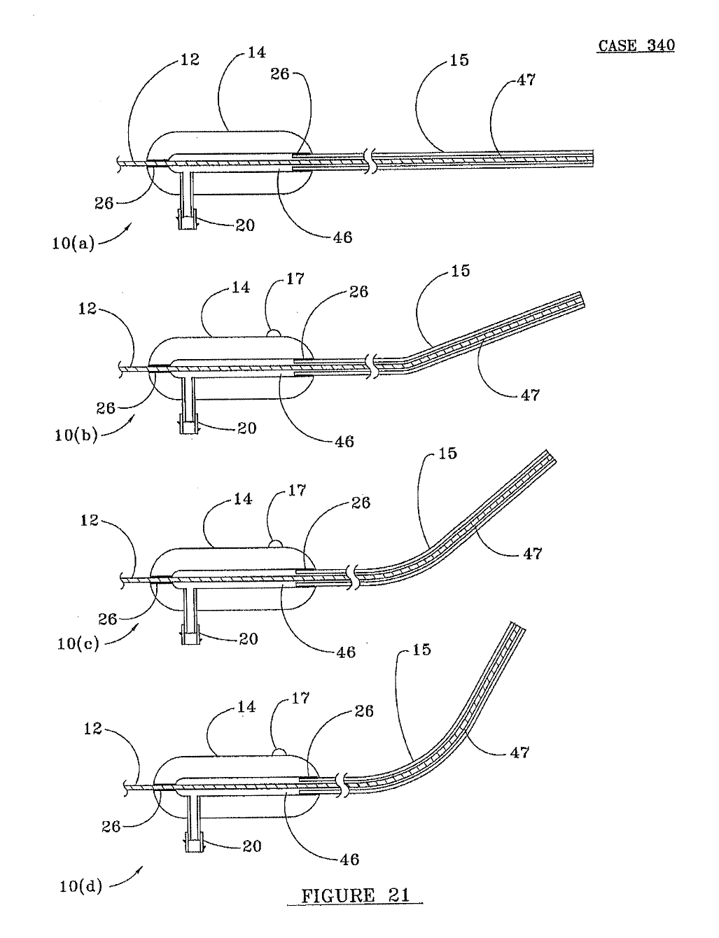

FIG. 21 illustrates an alternate embodiment of FIG. 20. In this embodiment of the present invention, cannula 15 can be made of a thin, rigid metal, preferably medical grade stainless steel or nitinol, for use under x-ray guidance through a body orifice, hollow organ, surgically created passageway or in a laparoscopic procedure, positioned and guided by an endoscope (not separately shown), through which cannula 15 may be inserted, or the endoscope may be inserted through a separate puncture.

Alternatively, cannula 15 can be made of a thin, flexible biocompatible plastic (not separately shown), for use through a flexible, articulated endoscope (not separately shown) or an endoscope of which the distal 5 to 15 cm may be bent or articulated at a described angle by wires (not separately shown) extending from a handpiece (not separately shown) to the distal end of the endoscope.

Preferably, devices 10(a)-(d) are made of a flexible memory metal, such as nitinol, an alloy of about 56% nickel and about 44% titanium by weight, such as those made by Memry, Inc. of Bethel, Conn., which are heat treated to “remember” their heat treated shape, to which they return after being straightened-out, for example, by passing through the instrument channel of an endoscope. Some semi-rigid plastics may also retain the memory of their initially molded shape, and can be used in devices 10(a)-(d) of FIG. 21.

In the embodiments of devices 10(a)-(d) of the present invention shown in FIG. 21, luer fitting 20 is fixedly attached within the wall of handpiece 14 and is in fluid communication with hollow passageway 46 in handpiece 14, as described in FIG. 14, and is in fluid communication with the space between the exterior of optical fiber 12 and the interior of cannula 15, creating a confined flow passageway or channel 47, enabling a sterile, bio-compatible liquid, such as saline or water, or a cold or cryogenically cooled gas, such as CO2 or nitrogen, to be infused through channel 47 to clean and cool the distal end of optical fiber 12 and to cool the Target Nerve Tissue, concomitantly with the delivery of laser energy to Target Nerve Tissue.

Optical fiber 12 is fixedly attached within the proximal end of handpiece 14, the proximal end of cannula 15, is fixedly attached within the distal end of handpiece 14 and luer fitting 20 can be fixedly attached to and in fluid communication with passageway 46 in handpiece 14, and fluid channel 47, as shown in FIG. 21. Likewise, collar 52 as described in FIG. 15, can be used to support and prevent damage to luer fitting 20, if luer fitting 20 is attached to cannula 15, as described above.

As can be seen, devices 10 (a)-(d) of FIG. 21 have bends at the same angles as devices 10(a)-(d) of FIG. 20. Again, such bends and others at any other desired angles may be employed, subject to the bend radius limitation described above.

The embodiments of devices 10 (a)-(d) of the present invention shown in FIG. 21 can be used in the Stationing, Moving, Rotating and/or Sweeping processes described above, individually or in any combination or sequence. The use of devices 10(a)-(d) shown in FIG. 21 are beneficial in instances where the use of side firing devices 10 of the present invention shown in FIG. 1-4, 6-8, 10 or 12 is difficult or considered impractical.

Alternatively, luer fitting 20 may be attached to cannula 15, distal to handpiece 14, as described in FIGS. 14 and 15, and luer fitting 20 may optionally be supported by collar 52, as described in FIG. 15.

All of the side firing devices of the present invention described in FIGS. 1-4, 6-8, 10 and 12 may be utilized with or without rigid plastic or metal cannula 15, with or without double-walled, hollow tube 33, or with or without collar 52 to support luer fitting 20. These appurtances, and the thinner walled capillary tube 29 of FIG. 4, are to enable any or all of the above embodiments of the present invention to better accomplish their desired purpose.

While the laser energy emission pattern 53 and laser energy spot area 31 of FIGS. 1-4, 6-8, 10, 12, 14-19, 20 and 21 are described as resulting from the emission of laser energy, any other thermal energy delivery device may be used in any of the above-described processes of Stationing, Moving, Rotating and/or Sweeping, alone or in any desired combination and in any desired sequence or order, to Interrupt S/PS/SN nerves of a Target Nerve Tissue to Treat a Medical Condition of a Patient.

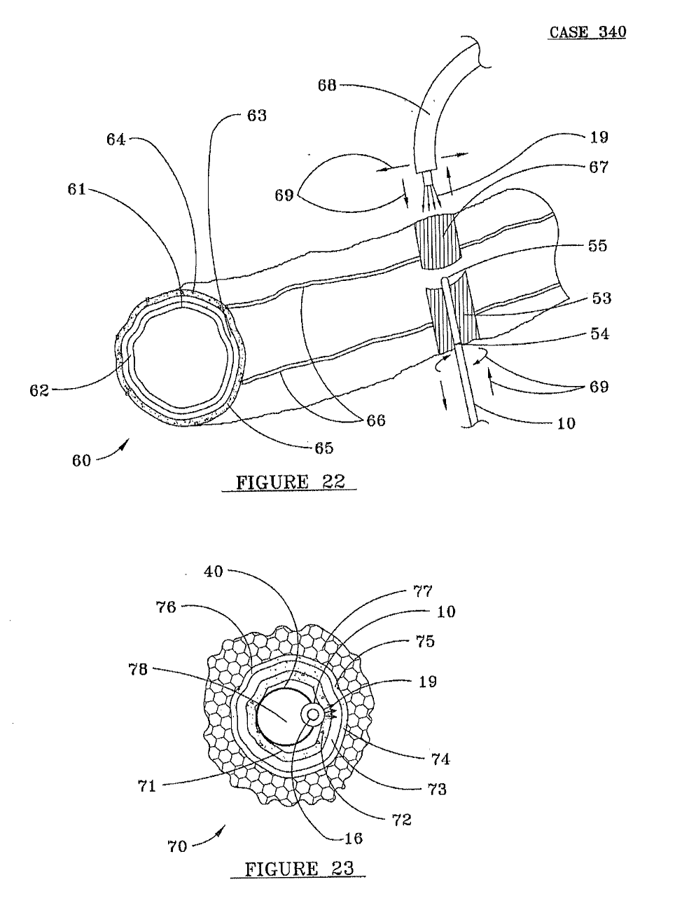

The uses of device 10 of FIGS. 1-4, 6-8, 10, 12, 20 and 21 of the present invention are shown in some of FIGS. 22-30 below and are intended to illustrate the methods of use of this invention to Interrupt S/PS/SN nerves to Treat a Medical Condition of a Patient. All of devices 10 illustrated in FIGS. 1-4, 6-8, 10, 12, 20 and 21 have a common purpose, namely to efficiently Interrupt malfunctioning S/PS/SN nerves of Target Nerve Tissues, when used by the methods of use described above.

FIG. 22 illustrates the cross sectional elements of a main renal artery 60, comprised of a very thin inner layer of epithelial cells 61, the intima muscle cell layer 62, the medial muscle cell layer 63, and the adventitia or outermost muscle cell layer 64 of renal artery 60. The adventitia layer 64 of renal artery 60 contains an aggregation of S/PS/SN nerves 65, and additional S/PS/SN nerves 66 are also seen attached to the exterior of renal artery 60. nerves 65 and 66 may malfunction and create hypertension, due to an unknown cause.

In addition to an intra-renal artery procedure, which can be performed by inserting a thermal energy delivery device, for example, any of the side firing laser devices of FIGS. 1-4, 6-8, 10 and 12, preferably the side firing devices of FIG. 10 or 12 with concentric, eccentric or back-mounted balloons 40, inserted through a conventional guiding catheter, from a puncture in the femoral artery in the groin, into the aorta and then into each of the main renal arteries of the kidneys to Interrupt S/PS/SN nerves 65 in the adventitia or outermost layer 64 and S/PS/SN nerves 66 on the exterior of renal artery 60.

However, it may be less traumatic to layers 61-63 of main renal artery 60 to Interrupt S/PS/SN nerves 65 in the adventitia or outermost layer 64 and S/PS/SN nerves 66 on the exterior of main renal artery 60 by applying a Source of Thermal Energy to S/PS/SN nerves 65 and 66 in a laparoscopic or endoscopic procedure from outside the main renal arteries 60. The benefit of Interrupting S/PS/SN nerves 65 and 66 in the outermost layer and on the exterior of the main renal arteries 60, respectively, is it avoids thermal damage to layers 61-63 of renal arteries 60, which may be denatured or coagulated and may subsequently become the locus of plaque deposits in the main renal arteries.

As shown in FIG. 22, rigid, side firing devices of FIGS. 1-4, 6 and 21 may be inserted in a laparoscopic procedure, with the abdomen distended by the infusion of CO₂ gas, and observed by a rigid endoscope which is inserted through a separate puncture. Intervening organs may be moved away by one or more rigid, blunt-ended obturators (not separately shown), which may be inserted through separate punctures, and devices 10 of FIGS. 1-4, 6 and 21 may be used to Interrupt malfunctioning S/PS/SN nerves 65 and 66 of the main renal arteries to treat hypertension in a laparoscopic procedure, as well as other Medical Conditions of a Patient.

Flexible, side firing devices of FIG. 1-4 or 6-8, and flexible devices 10(a)-(d) of FIGS. 20 and 21 may also be inserted into the abdomen through an articulated, flexible endoscope 68 or a rigid endoscope whose distal 5 to 15 cm may be flexible and articulated to likewise Interrupt such S/PS/SN nerves 65 in the outermost (adventitia) layer and S/PS/SN nerves 66 on the exterior of the main renal arteries to treat hypertension in an endoscopic procedure, as well as other Medical Conditions of a Patient.

Side firing devices of FIGS. 1-4 and 6 can be stationed, as shown, over the lower portion of the exterior of main renal artery 60. Device 10 is oriented to emit laser energy at 6 o'clock, and, while laser energy is being emitted, device 10 is Moved by advancing it from first point 54 to second point 55, while device 10 is Rotated through an arc of about 90° to 120°, from about 4 o'clock to 8 o'clock, in the Sweeping process described above, to Interrupt S/PS/SN nerves 65 in the adventitia or outermost layer 64 and S/PS/SN nerves 66 on the exterior of main renal artery 60, as shown by arrows 69.

Alternatively, as shown in the upper portion of the exterior of renal artery 60, flexible devices 10 of FIGS. 7-8, 20 and 21, wherein cannula 15 or sheath 34 is made of a memory metal or flexible plastic which retains its shape if bent, may be inserted through a flexible, articulated endoscope 68, or a rigid endoscope whose distal 5 to 15 cm is flexible and can be bent or articulated (not separately shown), in a retro-peritoneal procedure. This procedure is commonly used to remove and/or replace a kidney.

If device 10 is Stationed, Moved and/or Swept, as described above, creating irradiation area 67 to Interrupt a swath of S/PS/SN nerves 65 and 66. Also, irradiation area 67 can be enlarged by sequentially or simultaneously Moving and/or Sweeping articulated endoscope 68, as shown by arrows 69.

As described in co-owned U.S. Pat. No. 6,635,052 to Loeb, which is fully incorporated in its entirety herein by reference, one or more needles, with sharp or syringe-like distal ends (not separately shown), composed of a resilient material, such as a memory metal or nitinol, which, when straightened during passage through the instrument channel of an endoscope or a lumen of a rigid, semi-rigid, pliant or flexible cannula 15, resumes its initial bent shape, for example, of about 90°. Each needle contains an optical fiber and may be extended through layers 61-63 of renal artery 60 to Interrupt S/PS/SN nerves 65 in adventitia layer 64 and S/PS/SN nerves 66 on the exterior of renal artery 60, without subjecting layers 61-63 of renal artery 60 to damage from Thermal Energy which, for example, may subsequently become a locus for plaque formation.

If, for example, four such optical fiber containing needles are disposed within a flexible cannula 15 disposed within a main renal artery, are advanced out of cannula 15 and into the wall of the renal artery in an intra-luminal procedure at each of 12, 3, 6 and 9 o'clock, respectively, through layers 1-3 and, after laser energy is emitted at a desired level for a desired period of time, all four needles are retracted back into cannula 15.

To prevent the optical fiber containing needles to be inserted too deeply into a tissue containing S/PS/SN nerves, a flange may be attached proximal to the distal end of each optical fiber containing needle, to limit its insertion to a desired depth to achieve an optimal S/PS/SN nerve Interruption effect, without laser energy damaging intervening tissues.

Also, the optical fiber containing needles can be side firing, as described in FIGS. 1-4, 6-8, 10 and 12, emitting laser energy in a optimal manner, for example, in the second layer of the bronchi.

The ports or openings in the cannula through which the optical fiber needles, each with a flange, as described above, must be sufficiently large to allow the needles with flanges to exit and return into the cannula without interference.

The four needle containing cannula 15 may then be moved to a second position within the renal artery, and the needles are respectively inserted through layers 61-63 at 1, 4, 7 and 10 o'clock, after which the lasing and needle retraction procedures are repeated.

Then, after the four needle containing cannula 15 is moved to a third position, the needles are respectively inserted through layers 61-63 at each of 2, 5, 8 and 11 o'clock, and the lasing and retraction procedures are repeated. In three such positioning, insertion and lasing procedures, the S/PS/SN nerves of a main renal artery may be effectively and uniformly Denervated and Interrupted. Of course, any other number of optical fiber containing needles can be similarly used.

FIG. 23 illustrates the cross-sectional elements of a bronchi 70 of the lung, comprised in a staggered manner, each of a very thin inner layer of epithelial cells 71, a connective tissue layer 72, which contains an aggregation of S/PS/SN nerves 73, a muscle cell layer 74, an elastic fiber layer 75, which may also contain some S/PS//SN nerves 73, and an outer layer of mucous cells 76. While lumen 77 of the bronchi typically consists of multiple folds, lumen 77 in this drawing is shown expanded into a circle for simplicity of presentation.

In the treatment of asthma, for example, any of the side firing devices of FIGS. 1-4, 6-8, 10 and 12, preferably those of FIG. 10 or 12 for the reasons cited above, can be inserted through a flexible, articulated endoscope to Interrupt S/PS/SN nerves 73 in the second, connective tissue layer 72 of the bronchi 70, which may be sending incorrect signals to the brain to constrict muscle cell layer 74, due to an unknown cause, as well as to shrink or denature (changing the structure of certain proteins and damaging the DNA of the muscle cells in muscle cell layer 74, preventing the muscle cells from replicating) at a temperature of 50° C. to 60° C. or coagulating muscle cells in layer 74 at a temperature of 62° C. to 80° C., preventing or reducing the ability of muscle cell layer 74 to constrict the bronchi.

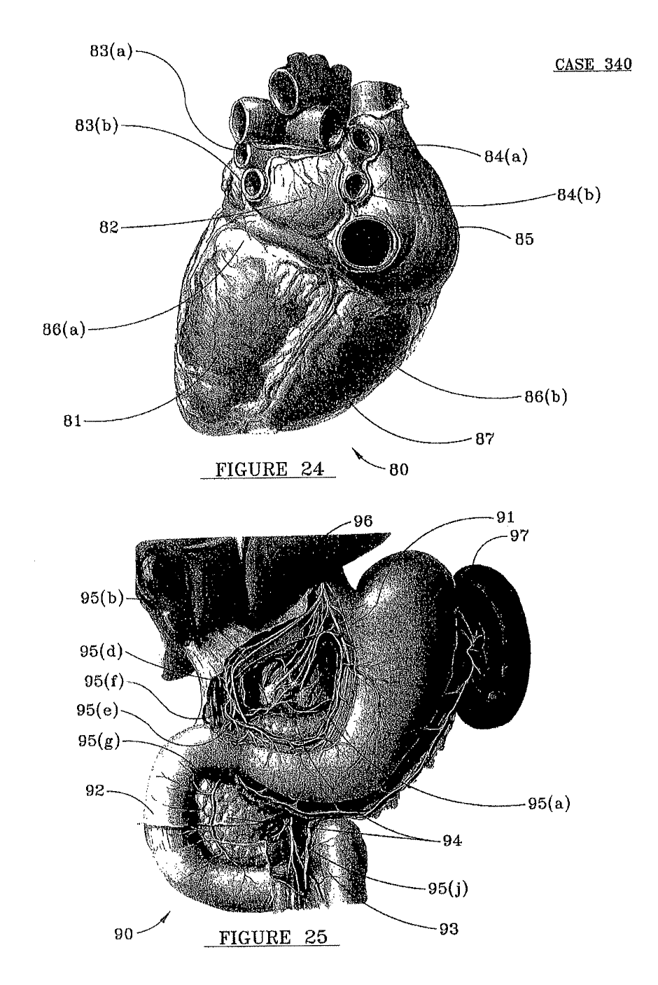

FIG. 24 illustrates an external, posteroinferior view of the heart 80, comprised of left ventricle 81, left atrium 82, left superior pulmonary vein 83(a), left inferior pulmonary vein 83(b), right superior pulmonary vein 84(a), right inferior pulmonary vein 84(b), right atrium 85, three fat pads 86(a-c) and right ventricle 87. Right pulmonary veins 84(a) and 84(b) contain an aggregation of S/PS/SN nerves (not separately shown) in their outermost layer or on their exterior of at least their distal 50 mm, where they join left atrium 82.

Also shown are fat pad 86(a), beneath left atrium 82, which contains the sinoatrial (“SA”) ganglion (not separately shown), fat pad 86(b), beneath right atrium 85, which contains the posterior atrial (“PA”) ganglion (not separately shown). Fat pad 86(c) on the anterior surface of the heart, beneath the left and right atria, 82 and 85, respectively, contains the anterior atrial (“AA”) ganglia of S/PS/SN nerves, which cannot be seen in this posteroinferior view of the heart. Other fat pads, may also contain a ganglia (not separately shown) of S/PS/SN nerves. The S/PS/SN nerves and their ganglia can be located within a fat pad by an electrical nerve stimulation device or electromyograph, as known in the art.

The use of device 10 of FIG. 1-4, 6-8, 10, 12, 20 or 21 to Interrupt S/PS/SN nerves in the distal 50 mm of right pulmonary veins 84(a) and (b), where they join left atrium 82 of the heart 80 can be accomplished intraluminally, preferably by the use of devices 10 of FIG. 10 or 12, most preferably with an eccentric or back mounted balloon 40 to bring laser energy emission port 19 close to the inner surface of veins 84(a) and (b). The cause of nerve malfunction creating arrhythmias is not known.

Alternatively, devices 10 of FIG. 1-4, 6-8, 20 or 21 can be used to Interrupt S/PS/SN nerves in the outermost layer and on the exterior of right pulmonary veins 84(a) and (b) from outside the heart in an endoscopic procedure, on a beating heart, with the endoscope inserted between the ribs and through the pericardial sac; or before or after coronary artery bypass graft (“CABG”) surgery with the chest open on a beating or on an arrested heart.

The Interruption of S/PS/SN nerves in the SA ganglion and the PA ganglion in fat pads 86(a) and (b), as well as the ganglia in Fat pad 86(c) (not separately shown) on the anterio-inferior surface of the heart, may reduce, suppress or eliminate cardiac arrhythmia, paroxysmal arrhythmia, bradyarrythmia, aterial fibrillation and the like.

FIG. 25 illustrates certain organs of the digestive system 90, including the stomach 91, duodenum 92, intestines 93, pancreas 94 and liver 96. Also shown are the gastric artery 95(a), the hepatic (liver) artery 95(b), the supraduodenal artery 95(c) the gastro-omental or gastroepiploic artery 95(d), the gastroduodenal artery 95(e), the pancreaticoduodenal artery 95(f), the esophageal branch of the gastric artery 95(g), the greater pancreatic artery 95(h), the colic artery 95(i) and the mesenteric artery 95(j).

These arteries 95(a)-(j) each contain an aggregation of S/PS/SN nerves and the ganglia associated with these nerves (not separately shown) can be accessed intraluminally from inside these arteries by inserting flexible devices 10 of FIG. 1-4, 6-8, 10 or 12, preferably flexible devices 10 of FIG. 10 or 12.

The cause of these S/PS/SN nerves malfunctioning, resulting in diabetes, Types I and II, insulin resistance, obesity and a variety of digestive disorders, is not known.

However, arteries 95(a)-(j) can also be Denervated and the S/PS/SN nerves on their exterior can be Interrupted from outside the arteries by the use of rigid devices 10 of FIG. 1-4, 6-8, 20 or 21 in a laparoscopic procedure, as described in FIG. 22, with the abdomen distended by the infusion of CO₂ gas. Alternatively, flexible devices 10 of FIG. 1-4, 6-8, 10, 12, 20 or 21 may be inserted through a fully or distally flexible, articulated endoscope to access the S/PS/SN nerves of arteries 95(a)-(j) from outside these arteries in an endoscopic procedure, as described in FIG. 22. If used without infusion of a cooling fluid, the laser power level should be about 0.5 to 3 watts, preferably 0.5 to 1 watts. If used with infusion of a cooling liquid, laser power level of about 3 to 20 watts, preferably about 5 to 10 watts, should be used.

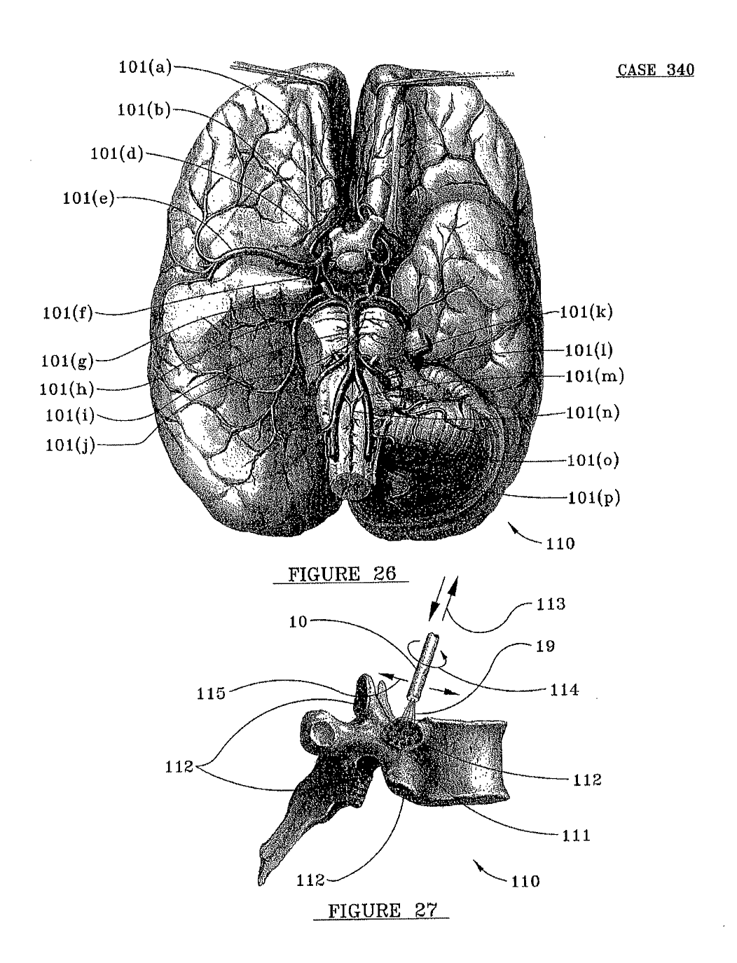

FIG. 26 illustrates the multiplicity of arteries of the brain 100 in an external, inferior view of brain 100. Shown are orbitefrontal (eye) artery 101(a), anterior cerebral artery 101(b), striate artery 101(c), the internal carotid artery 101(d), middle cerebral artery 101(e), anterior choroidal artery 101(f), posterior cerebral artery 101(g), superior cerebellar artery 101(h), basilar artery 101(i), pontine artery 101(j), labyrinthine artery 101(k), anterior cerebellar artery 101 (l), vertebral artery 101(m), anterior spinal artery 101(n), posterior cerebellar artery 101 (o) and posterior spinal artery 101(p). All of these arteries have an accumulation of S/PS/SN nerves, usually in their adventitia or outermost layer, on their exterior or running alongside their exterior (not separately shown).

The use of devices 10 of FIG. 1-4, 6-8, 10, 12, 20 or 21, as described heretofore, can Interrupt S/PS/SN nerves of arteries of the brain to Treat a variety of brain S/PS/SN nerve affected Medical Conditions in the brain or elsewhere in the body. These Medical Conditions can be Treated by Interrupting malfunctioning S/PS/SN nerves Within the walls of or on the exterior of the above arteries, based on known functions or determined by Evoked Potential testing, using an electromyograph. The S/PS/SN nerves of these arteries can be accessed by devices 10 of FIG. 1-4, 6-8, 10, 12, 20 or 21 through an endoscope, which is inserted through a surgically-created passageway or, in some cases, intraluminally.

These Medical Conditions include, among others, brain effected autism, Alzheimer's disease, senile dementia and others, psychological Medical Conditions, including schizophrenia (SOP), severe depression and others and brain effected bodily Medical Conditions, such as epilepsy, Parkinson's Disease and others, the cause of which is unknown.

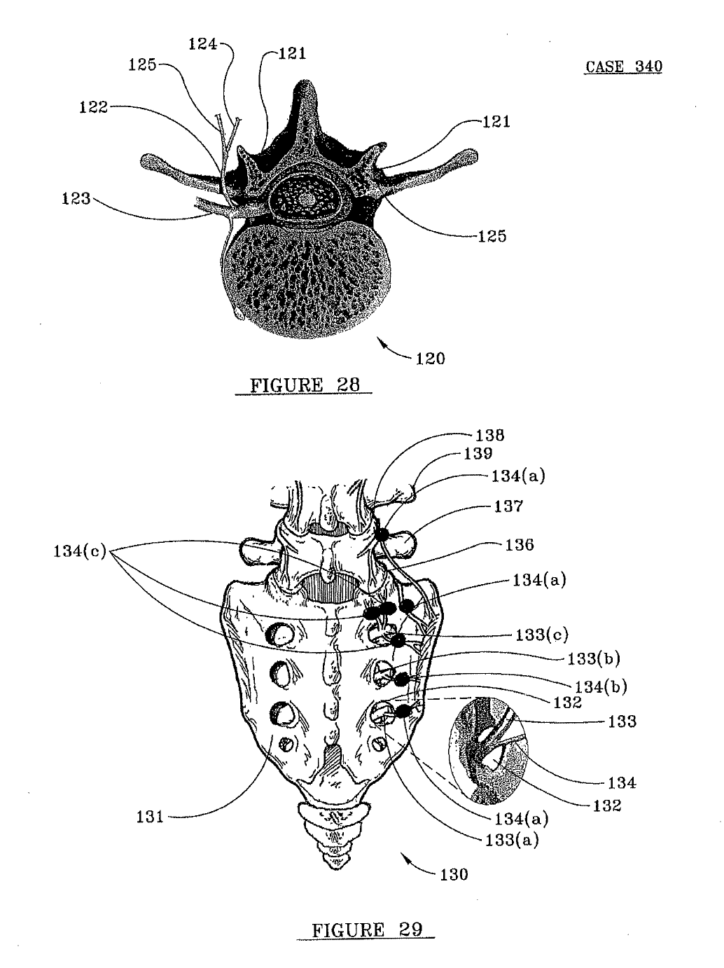

FIG. 27 illustrates an external view of a vertebra 110 of the spine, comprised of body 111, upper and lower facet joint surfaces 112, which matchingly fit with upper and lower facet joint surfaces 112 of vertebra 110 above and below vertebra 110. The cause of spinal S/PS/SN nerve malfunction is not known.

As shown, rigid devices of FIG. 1-4 or 6-8 or rigid devices 10(a)-(d) of FIG. 20 or 21, can be inserted under x-ray imaging, and laser energy, as shown by arrows 19, is emitted while any of devices 10 described above are Moved, Rotated and/or Swept, as determined by the surgeon, as shown by arrows 113, 114 and 115, respectively, to Interrupt tiny S/PS/SN nerve endings (not separately shown) in facet joint surface 112. After which, tiny S/PS/SN nerve endings (not separately shown) of the other facet joints 112 are similarly interrupted.

Alternatively, flexible or rigid devices of FIG. 1-4 or 6-8 or flexible or rigid devices 10(a)-(d) of FIG. 20 or 21 can be used through a-hollow metal cannula or an endoscope (not separately shown) to Interrupt tiny S/PS/SN nerve endings (not separately shown) in each of facet joint surfaces 112. Again, laser energies and power levels appropriate to the concomitant infusion of a cooling liquid or the infusion of no cooling liquid should be used.

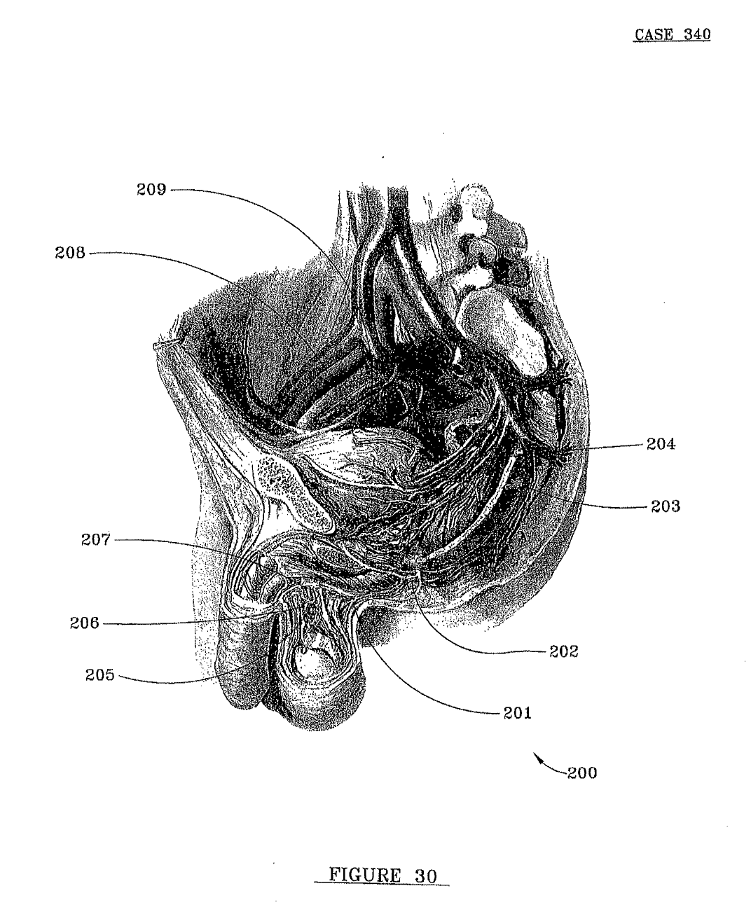

As illustrated in FIG. 28, an alternate method of treating back pain arising from the facet joints is described. Vertebra 120, facet joint surfaces 121 and dorsal nerve root 122 are shown. As an alternative to Interrupting the many tiny S/PS/SN nerve endings (not separately shown) in facet joint surfaces 121, some of which may be missed, the Interruption of dorsal nerve root 122 and/or the Interruption of medial branch 123 and lateral branch 124 of dorsal nerve root 122, using any of devices 10 of FIG. 1-4 or 6-8 or devices 10(a)-(d) of FIG. 20 or 21 to vaporize, by Positioning, Moving, Rotating or Sweeping, a sufficient length of the nerve to end the transmission of pain signals to the brain, as S/PS/SN nerve endings (not separately shown) in facet joint surfaces 121 communicate with the brain by transmission of nerve signals through dorsal nerve root 122 and its medial 123 and lateral 124 branches. Pain originating in the facet joint surfaces 121 is said to represent about 10% or more of back and neck pain complaints. Vertebral nerve 126 is also shown.

Alternatively, electro-shock wave (“ESW”) energy may be applied to fragment and destroy dorsal nerve root 122 and/or medial branch 123 and lateral branch 124 of dorsal nerve root 122 to Interrupt their transmission of pain signals to the brain.

FIG. 29 illustrates sacroiliac joint 130, comprised of the sacrum 131 of the spine, which in the expanded view shows portals 132, vertebral nerve 133 and sinu-vertebral nerve 134. Sacrum 131 overlays the two ilia of the hips.

As seen, vertebral nerve 133(a) extends from sacral joint S1 through portal 132 of sacrum 131, vertebral nerve 133(b) extends from sacral joint S2 through portal 132 of sacrum 131, vertebral nerve 133(a) extends from sacral joint S3 through portal 132 of sacrum 131. Sinu-vertebral nerve branches 134(a)-(e) extend from vertebral nerves 133 (a)-(c) above portals 132 of sacrum 131.

To treat pain arising from the sacroiliac joint 130, Holmium laser energy at a power level of about 3 to 10 watts, preferably about 5 watts, at a pulse repetition rate of about 10 pulses per second, is emitted for about 1 to 5 seconds, preferably about 2.5 seconds, to Interrupt each sinu-vertebral nerve 134(a)-(e) of S13 and L5 and 4.

Thermal Energy, for example, laser energy, preferably Holmium laser energy, may be transmitted through any of rigid or flexible devices 10(a)-(d) of FIG. 20 or 21, preferably devices 10(a)-(d) of FIG. 21, because they contain a fluid channel to infuse sterile water or saline to cool the tissue. Devices 10(a)-(d) of FIG. 20 or 21 can be used to Position, Move or Sweep the laser energy beam to Interrupt all eight (8) sinu-vertebral nerve branches 134(a)-(e) on one side of sacrum 131 and all eight (8) sinu-vertebral nerve branches 134(a-e) on the opposite side of sacrum 131.

The laser vaporization area is shown by the black dots. Laser energy is emitted to Interrupt sinu-vertebral nerve branch 134(a) of sacral joint 135, in this case, called S1. Laser energy is emitted to Interrupt sinu-vertebral nerve branch 134(b) of sacral joint 135, in this case, called S2. Laser energy is emitted to Interrupt three (3) sinu-vertebral nerve branches 135(c) of sacral joint 135, in this case, called S3.

Laser energy is also emitted to Interrupt sinu-vertebral nerve branch 135(d) of vertebral joint 136 of vertebra 137 and sacrum 131, in this case, called L5/S1, and laser energy is emitted to Interrupt two (2) sinu-vertebral nerve branches 135(e) of vertebral joint 138 of vertebra 137 and 139, in this case, called L4/L5.

Then, laser energy is emitted, on the opposite side of sacrum 131 and vertebra 137 and 139, to Interrupt the same sinu-vertebral nerve branches 134(a)-(e) on sacral joints 135 and vertebral joints 136 and 138. This ends the transmission of pain signals to the brain from sinu-vertebral nerve branches 134(a)-(e).

Rigid device 10(a) of FIG. 20 or 21, preferably rigid device 10(a) of FIG. 21, as it has a fluid channel to enable the infusion of a fluid, such as sterile water or saline, can be inserted under x-ray guidance or through a rigid endoscope to locate and apply laser energy by Positioning, Moving, Rotating or Scanning to Interrupt sinu-vertebral nerve branches 134(a)-(e) on both sides of the sacrum 131 and vertebra 137 and 139. Pain arising from sacroiliac joint 130 is said to represent about 20% or more of back pain complaints. The cause of sinu-vertebral nerve malfunction is not known.

Alternatively, electro-shock wave (“ESW”) energy may be focused at the points indicated by the black dots shown in FIG. 29 to fragment, destroy and Interrupt sinu-vertebral nerve branches 134(a)-(e).

FIG. 30 illustrates the male testes 200 and their arteries and veins, including the scrotal 201, perineal 202, pudendal 203, vesical 204, ductus deferens 205, papiniform 206 and testicular arteries 207, which contain in their outer layer or on their exterior an accumulation of S/PS/SN nerves. Also shown are a branch of vena cava 208 and umbilica artery 209, which contain an accumulation of S/PS/SN nerves.

Any of flexible or rigid side firing devices of FIG. 1-4 or 6-8 or devices 10(a)-(d) of FIG. 20 or 21, preferably devices 10(a)-(d) of FIG. 21, as the devices have space for infusion of a cooling fluid. These devices may be used through a rigid or flexible endoscope, as described heretofore, to Interrupt the S/PS/SN nerves of these arteries and branches of the vena cava, which control the rate of production, maturation and release of sperm to Treat male infertility, and such nerves often malfunction due to an unknown cause.

While not separately shown, the arteries of the hypothalamus and the female ovaries contain in their outer layer or on their exterior an accumulation of S/PS/SN nerves. Any of side firing devices of FIG. 1-4 or 6-8 or devices 10(a)-(d) of FIG. 20 or 21, preferably devices 10(a)-(d) of FIG. 21, for the reason set forth above, can be used, in the methods described heretofore, to Interrupt the S/PS/SNN nerves of these arteries, which control the rate of production, maturation and release of eggs to Treat female infertility. Such nerves often malfunction, creating female infertility, due to an unknown cause.

While this invention is susceptible of embodiment in many different forms, these are shown in the drawings and will be described in detail herein specific embodiments thereof, with the understanding that the present disclosure is to be considered as an exemplification of the principles of the invention and is not to be limited to the specific embodiment illustrated.

Numerous variations and modifications of the embodiments described above can be effected without departing from the spirit and scope of the novel features of the invention. It is to be understood that no limitation with respect to the specific apparatus illustrated herein is intended or should be inferred. It is, of course, intended to cover by the appended claims, all such modifications as fall within the scope of the claims.

I claim:

1. An apparatus for delivering laser energy to a Target Nerve Tissue comprised of an optical fiber inside a cannula and defining a confined flow passageway for delivery of a sterile, biocompatible liquid to cool and clean debris from a distal end portion of the optical fiber and to cool the Target Nerve Tissue.

2. The apparatus in accordance with claim 1 wherein distal end portion of the cannula is bendable to an angle of up to 90 degrees with respect to the rest of the cannula.

3. The apparatus in accordance with claim 1 wherein distal end portion of the cannula is pliant.

4. The apparatus in accordance with claim 1 wherein distal end portion of the cannula is flexible.

5. The apparatus in accordance with claim 1 wherein the cannula is made of a nickel titanium alloy.

6. The apparatus in accordance with claim 1 wherein the optical fiber has a core diameter of no more than 350 microns and has a beveled distal end portion.

7. A side firing optical fiber device which comprises
a cannula defining a side port;
an optical fiber within the cannula and defining therebetween a confined flow passageway for delivery of a biocompatible cooling liquid to irradiated tissue;
the optical fiber having an end portion encased in a closed-ended capillary tube and beveled for emission of laser energy laterally relative to the longitudinal axis of the optical fiber and through said side port of the cannula;
the device being sized for use through one of: an endoscope, a laparoscope and a surgically created passageway to treat malfunctioning S/PS/SN nerves within a blood vessel from the outside of the blood vessel, and through an introducer catheter into the lumen of a blood vessel to treat malfunctioning S/PS/SN nerves from the inside of the lumen of the blood vessel.

8. The side firing optical fiber device of claim 7, wherein a double-walled, multi-channel plastic tube is attached to the cannula for delivery of a sterile, biocompatible fluid to a least one of: (a) cool and clean debris from the tip of the side firing device and cool the Target Nerve Tissue; (b) inflate a balloon to close the lumen of a blood vessel; (c) inflate a balloon to press the laser energy emitting surface of the side firing optical fiber device close to the Target Nerve Tissue; and (d) enable excess fluid to flow from the balloon to one of: a drain and a collection bottle.

9. The side firing optical fiber device of claim 7, wherein said balloon is capable of venting excess fluid from the balloon.

10. A side firing optical fiber device sized for directing laser energy onto a Target Nerve Tissue to Treat a Medical Condition which comprises an optical fiber having core diameter of no more than 350 microns and a beveled end portion enveloped by a pliant sheath provided with a side port in alignment with laser energy emitted by the optical fiber.

11. The side firing optical fiber device of claim 10 defining a cooling fluid channel between the optical fiber and the sheath.

12. The side firing optical fiber device of claim 10 having a distal end of the optical fiber beveled at an angle in the range of about 35 degrees to about 45 degrees from the longitudinal axis of the optical fiber.

13. The side firing optical fiber device of claim 10 having a distal end of the optical fiber beveled at an angle in the range of about 40 degrees to about 41 degrees with respect to the longitudinal axis of the optical fiber.

14. The side firing optical fiber device of claim 10 wherein the beveled end portion comprises a pair of opposed beveled end surfaces each at an angle of 40 to 41 degrees from the longitudinal axis of the optical fiber.

15. The side firing optical fiber device of claim 10 wherein the beveled end portion is encased within a closed-ended capillary tube.

16. The side firing optical fiber device of claim 10 wherein the sheath is memory metal.

17. The side firing optical fiber device of claim 10 wherein the sheath is plastic.

18. A flexible optical fiber device bendable up to about 60 degrees from the longitudinal axis thereof and adapted to direct laser energy onto a Target Nerve Tissue to treat a Medical Condition and defining a channel between the optical fiber and a surrounding cannula for infusion of a biocompatible fluid for cooling the Target Nerve Tissue.

19. A method for Treating a Medical Condition of a Patient comprising of at least one of: Stationing, Moving, Rotating and Sweeping onto a Target Nerve Tissue a Thermal Energy in an amount sufficient to interrupt S/PS/SN nerves, including at least one of:
(a) the vagus and splanchnic nerves, within at least one of: (i) the esophagus, as well as the esophageal artery and vein of the esophagus; (ii) the hepatic artery and veins of the liver; (iii) the pancreas, as well as the pancreatic and pancreaticoduodenal arteries and their respective veins of the pancreas; (iv) the stomach, as well as the abdominal celiac and gastroepiploic arteries and their respective veins of the stomach; (v) the duodenum, as well as the celiac, gastroduodenal and pancreaticoduodenal arteries, their respective veins, and the portal vein of the duodenum; and (vi) the intestines, as well as the mesenteric, colic and sigmoid arteries and their respective veins of the intestines, the Medical Condition of the patient being at least one of: type II diabetes mellitus, insulin resistance, atherosclerosis (by changing the manner in which fat from the diet and fat released from stored fat is metabolized), obesity by at least one of: affecting the sensation of satiety, the manner in which fat from the diet and fat released from stored fat is metabolized and the means by which the volume of fat stored is controlled, ulcers, irritable bowel syndrome, celiac disease and other S/PS/SN nerve-affected, digestive system-related disorders;
(b) the S/PS/SN nerves within at least one of: the major arteries of the heart, the major veins of the heart and at least one of the fat pads of the heart and their associated ganglia, preferably the last 50 mm of the right pulmonary veins at their junction with the left atrium and the three fat pads of the heart located (i) on the posteroinferior surface of the heart beneath the left atrium containing the SA ganglion, (ii) on the posteroinferior surface of the heart beneath the right atrium containing the PA ganglion and (iii) on the anterior inferior surface of the heart beneath the left and right atria containing the AA ganglion, the Medical Condition of the patient being at least one of: arrhythmia, paroxysmal arrhythmia, bradyarrythmia, bradycardia, reduced myocardial contractibility, atrial fibrillation, ventricular fibrillation, cardiac arrest, chronic hear failure, acute heart failure, acute myocardial infarction, stroke, sleep apnea and other S/PS/SN nerve-affected, cardiac-related disorders;
(c) the vagus and splanchnic nerves, within at least one of: the popliteal, tibial, ilium, sacral and peroneal arteries and their respective veins and the saphenous vein of at least one of: hip, legs, and feet, the Medical Condition of the patient being at least one of: peripheral neuropathy, chronic leg cramps, claudication, edema, gangrene, restless leg syndrome and other S/PS/SN nerve-affected, peripheral (below the waist) artery-related disorders;
(d) the carotid, vagus and cervical nerves, within at least one of: the carotid and vertebral arteries and their respective veins of the brain, the Medical Condition of the patient being at least one of: epilepsy, seizures, convulsions, fragile X syndrome, autism, multiple sclerosis, amyotrophic lateral sclerosis (ALS), myasthenia gravis, severe depression, migraine headaches, schizophrenia, psychosis, anxiety, Parkinson's disease, Huntington's disease, senile dementia, Alzheimer's disease and other S/PS/SN nerve-affected, central nervous system-related disorders;
(e) the carotid, vagus and cervical nerves, within at least one of: the basilar, carotid, cerebral and caudate arteries and their respective veins and the thalomostrate vein of the thalamus, the Medical Condition of the patient being at least one of: insomnia, narcolepsy, multiple sclerosis, amyotrophic lateral sclerosis (ALS), myasthenia gravis, the need to at least one of: attract stem cells to repair tissue, attract killer white cells to attack at least one of bacteria, a virus or cancer cells (by affecting the means by which the volume, rate of maturism and release of stem cells and white cells is controlled) and other S/PS/SN nerve-affected. thalamus-related disorders;
(f) the cervical, carotid and vagus nerves, within at least one of: the basilar, cerebral, hypothalamic, carotid and caudate arteries and their respective veins of the hypothalamus and the pituitary and hypophyseal arteries and their respective veins of the pituitary gland, the Medical Condition of the patient being at least one of: hypogonadism, hypothyroxinemia (low concentration of thryroxine in the blood stream), hyperthyroidism (high concentration of thyroxin in the blood stream), infertility, irregular menorrhagia, Type II diabetes mellitus and other S/PS/SN nerve-affected hypothalamus and pituitary gland-related disorders;
(g) the vagus, cervical, spinous and sciatic nerves of the vertebra, the nerve endings in the capsule of the facets, dorsal primary ramus nerves of the facets and the sacral and sinu-vertebral nerves of the sacrum, within at least one of: (i) the vertebra, a spinal disc, the sacrum, the hips, the long bones of the arms and legs and the small bones of the ankles, feet, toes, wrists, hands and fingers; (ii) the vertebral, spinal and intercostal arteries, their respective veins and the vena cava vein of the vertebra; (iii) the vertebral and sacral arteries and their respective veins of the sacrum; (iv) the subclavian, brachial, humeral and scapular arteries and their respective veins of the joints of the shoulders; (v) the radial and ulnar arteries and their respective veins of the joints of the elbows; (vi) the palmar and digital arteries and their respective veins of the joints of at least one of: the wrists, hands and fingers; (vii) the femoral artery and vein and saphenous vein of the joints of the hips; (viii) the femoral, ilium and popliteal arteries and their respective veins and the saphenous vein of the joints of the knees; and (ix) the popliteal, tibial and peroneal arteries and their respective veins and the saphenous vein of the joints of at least one of: the ankles, feet and toes, the Medical Condition of the patient being pain arising from at least one of: arthritis, avascular necrosis, osteonecrosis, osteocarcoma and physical damage of at least one of: the vertebra, spinal discs and bones; and pain arising from the joints of at least one of: the vertebra, facets, the joint of the sacrum and ilium, shoulders, elbows, wrists, hands, fingers, hips, knees, ankles, feet and toe and other S/PS/SN nerve-affected, joint-related disorders;
(h) the vagus and splanchnic nerves, within at least one of: the splenic arteries and veins of the spleen, the Medical Condition of the patient being one of: hypohemoglobia (anemia) and hyperhemoglobia (by affecting the means by which the volume and rate of destruction of red cells is controlled) and other S/PS/SN nerve-affected, spleen-related blood disorders;
(i) the vagus and splanchnic nerves, within at least one of: the uterine arteries and veins of the uterus, the Medical Condition of the patient being at least one of: uterine cramps, hot flashes, amenorrhea, dysmenorrhea and infertility (to affect the means by which the volume, type and rate of production of reproductive hormones are controlled) and other S/PS/SN nerve-affected, uterine-related disorders;
(j) the vagus and splanchnic nerves, within at least one of: the ilium, uterine and ovarian arteries and their respective veins of the ovaries, the Medical Condition of the patient being infertility by affecting the means by which the maturity of eggs, the number of eggs released and their rate of release is controlled, menopause by affecting the means by which the type, volume and rate of production of reproductive hormones is controlled and other S/PS/SN nerve-affected, ovarian-related disorders;
(k) the pelvic and splanchnic nerves, within at least one of: the ilium, epigastric and testicular arteries and their respective veins of the testes, the Medical Condition of the patient being at least one of: aspermia (male infertility), by affecting the means by which the volume and maturity of sperm produced and released is controlled and hypogonadism by affecting the means by which the volume and rate of production of testosterone and its release is controlled and other S/PS/SN nerve-affected, testis-related disorders;
(l) the pelvic and splanchnic nerves, within at least one of: the pudental arteries and veins of the penis, the Medical Condition of the patient being at least one of male impotence by affecting the means by which blood flow to cause erection of the penis is controlled and maintained and other S/PS/SN nerve-affected, penile-related disorders;
(m) the pelvic and splanchnic nerves, within at least one of: the rectal artery and vein of the rectum and anus, the Medical Condition of the patient being fecal incontinence and other S/PS/SN nerve-affected, rectal and anal-related conditions;
(n) the frontal and parietal branches of the superficial arteries and veins of the scalp, the Medical Condition of the patient being at least one of: alopecia (baldness), by affecting the means by which at least one of: the type and volume of testosterone is produced and its rate of release is controlled, and dandruff, by affecting the means by which the volume of blood delivered to the scalp and its rate of flow is controlled, and other S/PS/SN nerve-affected, scalp-related disorders; and
(o) the S/PS/SN nerve endings in the capsule and the medial branches of the primary dorsal ramus nerve of the facet joints and the vertebral, sinu-vertebral, vagus and splanchnic nerves of the sacroiliac joint of the sacrum and ilium, preferably the sine-vertebral nerves, and their branches in the joints of at least the two vertebra above the sacrum; the Medical Condition of the patient being at least one of: neck, shoulder, back, hip and leg pain originating in the facets of the vertebra and the sacroiliac joint of the sacrum and ilium and their branches in the joints of at least the two vertebra above the sacrum; and other S/PS/SN nerve-affected, facet and sacroiliac joint-related disorders.

20. The method of claim 19, wherein the Source of Thermal Energy is preferably a CTH:YAG laser, which interrupts S/PS/SN nerves, tissues containing S/PS/SN nerves, and other Target Nerve Tissues, with less damage to adjacent tissues; as it allows significant time between pulses of laser energy for the tissue to cool.

21. The method of claim 19, wherein the laser energy is delivered to a Target Nerve Tissue by one of: (a) multiple beams of laser energy focused to intersect at a desired point and (b) a beam of laser energy focused to converge at a desired point, the point being about 2 mm to 5 mm from the laser energy emitting surface of the device, to shrink a Target Tissue.

22. A method for Treating a Medical Condition of a Patient comprising of at least one of: Stationing, Moving, Rotating and Sweeping Onto a Target Nerve Tissue a Source of one of: pulsed laser energy and continuous wave laser energy, delivered at a desired angle of one of: an angle of 0°, 1° to 60°, and 61° to 90° from the axis of laser beam exiting the Source of laser energy, through at least one needle containing an optical fiber to a desired depth within a Target Nerve Tissue and multiple beams of at least one of: laser, x-ray, proton, RF, MW and US energy, focused to intersect at a desired point, to interrupt S/PS/SN nerves within the renal arteries of the kidneys, preferably the left and right main renal arteries, the Medical Condition of the patient being at least one of: hypertension (systolic blood pressure equal to or greater than 140 mm of mercury), chronic heart failure, acute heart failure, acute myocardial infarction, stroke, insulin resistance, type II diabetes mellitus, sleep apnea and other S/PS/SN nerve-affected, kidney-related disorders.

23. A method for Treating a Medical Condition of a Patient comprising of at least one of: Stationing, Moving, Rotating and Sweeping Onto a Target Nerve Tissue a Source of one of: pulsed laser energy and continuous wave laser energy, delivered at one of: a desired angle of at least one of: 0°, 1° to 60°, and 61° to 90° from the axis of laser beam exiting the source of laser energy, through at least one needle, containing an optical fiber to a desired depth within a Target Nerve Tissue and multiple beams of at least one of: laser, x-ray, proton, RF, MW and US energy, focused to intersect at a desired point, to at least one of: interrupt S/PS/SN nerves, including the lower trachea and branches of the bronchi, as well as the vagus nerve, and after a layer of muscle cells within the bronchi of the lungs, the Medical Condition of the patient being at least one of: asthma, chronic obstructive pulmonary disease and other S/PS/SN nerve and muscle cell-affected, pulmonary-related disorders.
